(19) United States
(12) Patent Application Publication
(10) Pub. No.: US 2006/0270742 A1
(43) Pub. Date: Nov. 30, 2006

(54) Compositions and methods for the treatment of neurodegenerative diseases

(76) Inventors: Jane Staunton, Roslindale, MA (US); Xiaowei Jin, Cambridge, MA (US); Dina Solimini Rufo, South Weymouth, MA (US); Michael Monteiro, Malden, MA (US)

Correspondence Address: CLARK & ELBING LLP, 101 FEDERAL STREET, BOSTON, MA, 02110, US

(21) Appl. No.: 11/415,786

(22) Filed: May 2, 2006

Related U.S. Application Data
(60) Provisional application No. 60/677,022, filed on May 2, 2005.
(60) Provisional application No. 60/698,184, filed on Jul. 11, 2005.
(60) Provisional application No. 60/761,573, filed on Jan. 24, 2006.

(51) Int. Cl. A61K 31/13

(52) U.S. Cl. 514/663; 514/474; 514/571

(57) ABSTRACT

The invention features compositions and methods for the treatment of neurodegenerative diseases.

### CROSS-REFERENCE TO RELATED APPLICATIONS

This application claims benefit from U.S. Provisional Application Nos. 60/677,022, filed May 2, 2005, 60/698,184, filed Jul. 11, 2005, and 60/761,573, filed Jan. 24, 2006, each of which is hereby incorporated by reference.

### BACKGROUND OF THE INVENTION

Proximal Spinal Muscular Atrophy (SMA), a common genetic cause of infant mortality, is an autosomal recessive disorder in which alpha motor neuron death in the spinal cord is observed. The primary genetic lesion that causes SMA is a deletion or mutation of the telomeric copy of the survival motor neuron gene (SMN1). The centromeric survival motor neuron gene (SMN2), a hypofunctional allele of SMN1, is unaffected in the disease. This information has lead to the generation of a mouse model of SMA, in which the single mouse SMN gene is deleted and the resulting embryonic lethality is suppressed by introduction of the human SMN2 transgene. SMN is a 38 kDa protein ubiquitously expressed in both cytoplasm and nuclei. In the nucleus, SMN is found in gemini of coiled bodies (gems), named for their association with coiled bodies. SMN associates with itself and forms a complex with a series of proteins, including the Sm proteins, SIP-1 (gemin 2), gemin 3 and gemin 4 and possibly other proteins. This SMN-containing complex functions in snRNP biogenesis, participating in pre-mRNA splicing in the nucleus. A series of other proteins have been reported to interact with SMN, including profilins, E2 and FUSE, suggesting other possible roles for SMN. Despite the insights derived from identification of SMN as the principal genetic cause of SMA, the detailed molecular pathogenesis of the disease remains enigmatic. The basis for selective death of alpha motor neurons compared to other cell types in SMA patients and mice is not understood.

The primary molecular defect in most patients with SMA is decreased SMN protein levels. This deficiency results in the selective degeneration of lower motor neurons and the loss of motor function, and is frequently fatal. Small molecules that increase the amount of SMN protein in cells are much sought after for their potential therapeutic value to SMA patients. Previous screens and research efforts have been directed towards discovering small molecules that alter splicing of the SMN2 pre-mRNA, or of compounds that activate the SMN2 promoter. However, many of these compounds do not increase the amount of SMN protein in cells by a significant amount. In addition, most of the identified compounds show toxicities that limit their therapeutic suitability.

Thus there is a need for agents that may be used to treat SMA and other neurodegenerative diseases.

### SUMMARY OF THE INVENTION

We have identified memantine and amantadine as two compounds that increase SMN levels in SMA patient fibroblasts in vitro. These compounds, or analogs thereof, may be used alone or in combination for the treatment of spinal muscular atrophy (SMA) or spinal and bulbar muscular atrophy (SBMA).

Accordingly, in one aspect, the invention features a method for treating a neurodegenerative disease or increasing SMN protein levels in a patient having SMA by administering to a patient in need thereof memantine, amantadine, or an analog thereof, alone or in combination with one or more agents selected from alosetron, amrinone, ascorbic acid, guanfacine, indoprofen, ubenimex, and agents useful for treating a neurodegenerative disease. If two or more agents are administered, it is desirable that the agents be administered simultaneously or within 28 days, 14 days, 10, days, 7 days, or 24 hour of each other, or simultaneously, in amounts that together are sufficient to treat the neurodegenerative disease or increase SMN protein levels. If the patient is administered more than one agent, the different agents may be admixed together in a single formulation or in separate formulations. If administered in separate formulations, the agents may or may not be administered by the same route of administration (e.g., oral, intravenous, intramuscular, ophthalmic, topical, dermal, subcutaneous, and rectal). If desired, an agent maybe administered at a high dosage or low dosage.

The invention also features a composition that includes: (a) memantine, amantadine, or an analog thereof; and (b) a second agent selected from alosetron, amrinone, ascorbic acid, guanfacine, indoprofen, ubenimex, and agents useful for treating a neurodegenerative disease. Desirably, the two agents are present in amounts that, when administered together to a patient, are sufficient to treat a neurodegenerative disease or increase SMN protein levels (i.e., result in a statistically significant increase in SMN protein levels compared to a control). The composition may be formulated for oral or systemic administration.

The invention also features kits for treating neurodegenerative diseases.

One such kit includes (i) an agent selected from memantine, amantadine, and analogs thereof; and (ii) instructions for administering the agent to a patient having a neurodegenerative disease, either alone or in combination with a second agent selected from alosetron, amrinone, ascorbic acid, guanfacine, indoprofen, ubenimex, and agents useful for treating a neurodegenerative disease.

Another such kit includes (i) a composition containing (a) memantine, amantadine, or an analog thereof; and (b) a second agent selected from alosetron, amrinone, ascorbic acid, guanfacine, indoprofen, ubenimex, and agents useful for treating a neurodegenerative disease; and (ii) instructions for administering the composition to a patient having a neurodegenerative disease.

Yet another kit includes (i) a first agent selected from memantine, amantadine, and analogs thereof; (ii) a second agent selected from alosetron, amrinone, ascorbic acid, guanfacine, indoprofen, ubenimex, and agents useful for treating a neurodegenerative disease; and (iii) instructions for administering the first and second agents to a patient having a neurodegenerative disease.

Still another kit includes (i) an agent selected from alosetron, amrinone, ascorbic acid, guanfacine, indoprofen, ubenimex, and agents useful for treating a neurodegenerative disease; and (ii) instructions for administering the agent and a second selected from memantine, amantadine, and analogs thereof to a patient having a neurodegenerative disease.

The invention also features a kit that includes memantine, amantadine, or an analog thereof and information about (i) SMA and/or (ii) how to administer a drug (e.g., memantine, amantadine, or an analog thereof) to children (e.g., children with SMA).

The invention also features a method of distributing memantine, amantadine, or an analog thereof directly to SMA patients through a patient registry.

The invention also features a method of identifying a combination that may be useful for the treatment of a neurodegenerative disease. This method includes the steps of: (a) contacting SMN-expressing cells with (i) an agent selected from memantine, amantadine, and analogs thereof; and (ii) a candidate compound; and (b) determining whether the combination of the agent and the candidate compound increase the amount of SMN protein relative to cells contacted with the agent but not contacted with the candidate compound, wherein an increasing in the amount of SMN protein identifies the combination as a combination useful for the treatment of a neurodegenerative disease. Desirably, the cells are mammalian cells (e.g., human fibroblasts from an SMA patient)

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

By “an amount sufficient” is meant the amount of a compound, alone or in combination with another therapeutic regimen, required to treat, prevent, or reduce a metabolic disorder such as diabetes in a clinically relevant manner. A sufficient amount of an active compound used to practice the present invention for therapeutic treatment of conditions caused by or contributing to diabetes varies depending upon the manner of administration, the age, body weight, and general health of the mammal or patient. Ultimately, the prescribers will decide the appropriate amount and dosage regimen. Additionally, an effective amount may be an amount of compound in the combination of the invention that is safe and efficacious in the treatment of a patient having a metabolic disorder such as diabetes over each agent alone as determined and approved by a regulatory authority (such as the U.S. Food and Drug Administration).

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that reduces glucose levels and that is formulated for administration by inhalation will differ from a low dosage of the same agent formulated for oral administration.

By a “high dosage” is meant at least 5% (e.g., at least 10%, 20%, 50%, 100%, 200%, or even 300%) more than the highest standard recommended dosage of a particular compound for treatment of any human disease or condition.

By a “candidate compound” is meant a chemical, be it naturally-occurring or artificially-derived. Candidate compounds may include, for example, peptides, polypeptides, synthetic organic molecules, naturally occurring organic molecules, nucleic acid molecules, peptide nucleic acid molecules, and components and derivatives thereof.

“Alkyl” refers to unsubstituted or substituted linear, branched or cyclic alkyl carbon chains of up to 15 carbon atoms (unless otherwise specified). Linear alkyl groups include methyl, ethyl, n-propyl, n-butyl, n-pentyl, n-hexyl, n-heptyl and n-octyl. Branched alkyl groups include iso-propyl, sec-butyl, iso-butyl, tertbutyl and neopentyl. Cyclic alkyl groups include cyclopropyl, cyclobutyl, cyclopentyl and cyclohexyl. Alkyl groups can be substituted with one or more substituents. Nonlimiting examples of such substituents include NO₂, ONO₂, F, Cl, Br, I, OH, OCH₃, CO₂H, CO₂ CH₃, CN, aryl and heteroaryl. Where “alkyl” is used in a context such as “alkyl-ONO₂,” it refers to an alkyl group that is substituted with an ONO₂ moiety. Where “alkyl” is used in a context such as “C(O)alkyl-ONO₂,” it refers to an alkyl group that is connected to a carbonyl group at one position and substituted with an ONO₂ moiety.

“Heteroalkyl” refers to unsubstituted or substituted linear, branched or cyclic chains of up to 15 carbon atoms that contain at least one heteroatom (e.g., nitrogen, oxygen or sulfur) in the chain. Linear heteroalkyl groups include CH₂CH₂OCH₃, CH₂CH₂N(CH₃)₂ and CH₂CH₂SCH₃. Branched groups include CH₂CH(OCH₃)CH₃, CH₂CH(N(CH₃)₂)CH₃ and CH₂CH(SCH₃)CH₃. Cyclic heteroalkyl groups include CH(CH₂CH₂)₂O, CH(CH₂CH₂)₂NCH₃ and CH(CH₂CH₂)₂S. Heteroalkyl groups can be substituted with one or more substituents. Nonlimiting examples of such substituents include NO₂, ONO₂, F, Cl, Br, I, OH, OCH₃, CO₂H, CO₂ CH₃, CN, aryl and heteroaryl. Where “heteroalkyl” is used in a context such as “heteroalkyl-ONO₂,” it refers to a heteroalkyl group that is substituted with an ONO₂ moiety. Where “heteroalkyl” is used in a context such as “C(O)heteroalkyl-NO2,” it refers to an alkyl group that is connected to a carbonyl group at one position and substituted with an ONO₂ moiety.

By “halo” means F, Cl, Br, or I.

The term “aryl” refers to an unsubstituted or substituted aromatic, carbocyclic group. Aryl groups are either single ring or multiple condensed ring compounds. A phenyl group, for example, is a single ring, aryl group. An aryl group with multiple condensed rings is exemplified by a naphthyl group. Aryl groups can be substituted with one or more substituents. Nonlimiting examples of such substituents include NO₂, ONO₂, F, Cl, Br, I, OH, OCH₃, CO₂H, CO₂ CH₃, CN, aryl and heteroaryl.

The term “heteroaryl” refers an unsubstituted or substituted aromatic group having at least one heteroatom (e.g., nitrogen, oxygen, or sulfur) in the aromatic ring. Heteroaryl groups are either single ring or multiple condensed ring compounds. Single ring heteroaryl groups having at least one nitrogen include, for example, tetrazoyl, pyrrolyl, pyridyl, pyridazinyl, indolyl, quinolyl, imidazolyl, isoquinolyl, pyrazolyl, pyrazinyl, pyrimidinyl and pyridazinonyl. A furyl group, for example is a single ring heteroaryl group containing one oxygen atom. A condensed ring heteroaryl group containing one oxygen atom is exemplified by a benzofuranyl group. Thienyl, for example, is a single ring heteroaryl group containing one sulfur atom. A condensed ring heteroaryl group containing one sulfur atom is exemplified by benzothienyl. In certain cases, heteroaryl groups contain more than one kind of heteroatom in the same ring. Examples of such groups include furazanyl, oxazolyl, isoxazolyl, thiazolyl, and phenothiazinyl. Heteroaryl groups can be substituted with one or more substituents. Nonlimiting examples of such substituents include NO₂, ONO₂, F, Cl, Br, I, OH, OCH₃, CO₂H, CO₂ CH₃, CN, aryl and heteroaryl.

Compounds useful in the invention include those described herein in any of their pharmaceutically acceptable forms, including isomers such as diastereomers and enantiomers, salts, esters, solvates, and polymorphs thereof, as well as racemic mixtures and pure isomers of the compounds described herein.

Other features and advantages of the invention will be apparent from the detailed description and from the claims.

### BRIEF DESCRIPTION OF THE DRAWINGS

FIG. 1 is a series of illustrations depicting absolute SMN fold induction of various drug combinations. SMN fold induction was calculated as SMN(T−B)/SMN(U−B) where “T” is the signal from treated cells, “B” is plate-specific background, and “U” is the signal from untreated cells.

FIG. 2 is a series of illustrations depicting viability-controlled fold induction of various drug combinations. Viability-controlled fold induction was calculated as (SMN fold induction)/(ATP fold induction), where ATP fold induction is calculated in the same manner as SMN fold induction, or: ATP(T−B)/ATP(U−B).

FIG. 3 is a graph showing viability-controlled SMN fold increase following exposure to various concentrations of memantine.

FIGS. 4-6 are illustrations showing memantine-induced increases in SMN protein relative to control proteins GAPdH and eIF4E.

### DETAILED DESCRIPTION

We have identified memantine (1-amino-3,5-dimethyl adamantane) and amantadine as agents that increase SMN protein levels in SMA fibroblasts in vitro. These agents may be used to increase SMN protein levels in patients having SMA or SBMA, and may further be used to treat these patients.

Memantine, Amantadine, and Analogs Thereof

Memantine analogs include compounds having the formula (I): 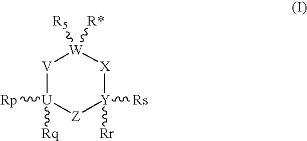
wherein R* is -(A)ₙ-(CR₁R₂)ₘ—NR₃R₄, n+m=0, 1, or 2, A is selected from the group consisting of linear or branched C₁-C₆ alkyl, linear or branched C₂-C₆ alkenyl, and linear or branched C₂-C₆ alkynyl, R₁ and R₂ are independently selected from the group consisting of hydrogen, linear or branched C₁-C₆ alkyl, linear or branched C₂-C₆ alkenyl, linear or branched C₂-C₆ alkynyl, aryl, substituted aryl, and arylalkyl, R₃ and R₄ are independently selected from the group consisting of hydrogen, linear or branched C₁-C₆ alkyl, linear or branched C₂-C₆ alkenyl, and linear or branched C₂-C₆ alkynyl, or together form C₂-C₁₀ alkylene or C₂-C₆ alkenylene or together with the N form a 3-7-membered azacycloalkane or azacycloalkene, including substituted (C₁-C₆ alkyl, C₂-C₆ alkenyl) 3-7-membered azacycloalkane or azacycloalkene; or independently R₃ or R₄ may join with Rₚ, R_{q}, Rᵣ, or Rₛ to form an alkylene chain —CH(R₆)—(CH₂)ₜ—, wherein t=0 or 1 and the left side of the alkylene chain is attached to U or Y, the right side of the alkylene chain is attached to N, and R₆ is selected from the group consisting of hydrogen, linear or branched C₁-C₆ alkyl, linear or branched C₂-C₆ alkenyl, linear or branched C₂-C₆ alkynyl, aryl, substituted aryl and arylalkyl; or independently R₃ or R₄ may join with R₅ to form an alkylene chain represented by the formula —CH₂—CH₂—CH₂—CH₂)ₜ—, or an alkenylene chain represented by the formulae —CH═CH—CH₂—(CH₂)ₜ—, —CH═C═CH—(CH₂)ₜ— or —CH₂—CH═CH—(CH₂)ₜ—, wherein t=0 or 1, and the left side of the alkylene or alkenylene chain is attached to W and the right side of the alkylene ring is attached to N; R₅ is selected from the group consisting of hydrogen, linear or branched C₁-C₆ alkyl (C₁-C₆), linear or branched C₂-C₆ alkenyl, and linear or branched C₂-C₆ alkynyl, or R₅ combines with the carbon to which it is attached and the next adjacent ring carbon to form a double bond, Rₚ, R_{q}, Rᵣ, and Rₛ, are independently selected from the group consisting of hydrogen, linear or branched C₁-C₆ alkyl, linear or branched C₂-C₆ alkenyl, linear or branched C₂-C₆ alkynyl, C₃-C₆ cycloalkyl, aryl, substituted aryl, and arylaklyl, or Rₚ, R_{q}, Rᵣ, or Rₛ independently may form a double bond with U or with Y or to which it is attached, or Rₚ, R_{q}, Rᵣ, or Rₛ may combine together to represent a lower alkylene —(CH₂)ₓ— or a lower alkenylene bridge wherein x is 2-5, inclusive, which alkylene bridge may, in turn, combine with R₅ to form an additional lower alkylene —(CH₂)_{y}— or a lower alkenylene bridge, wherein y is 1-3, inclusive, U, V, W, X, Y, Z represent carbon atoms, and include optical isomers, diastereomers, polymorphs, enantiomers, hydrates, pharmaceutically acceptable salts, and mixtures of compounds within formula (I).

The ring defined by U—V—W—X—Y-Z is preferably selected from the group consisting of cyclohexane, cyclohex-2-ene, cyclohex-3-ene, cyclohex-1,4-diene, cyclohex-1,5-diene, cyclohex-2,4-diene, and cyclohex-2,5-diene.

Examples of memantine analogs that can be employed in the methods, compositions, and kits of the invention include the memantine analogs selected from the group consisting of 1-amino-1,3,5-trimethylcyclohexane, 1-amino-1 (trans),3(trans),5-trimethylcyclohexane, 1-amino-1 (cis),3(cis),5-trimethylcyclohexane, 1-amino-1,3,3,5-tetramethylcyclohexane, 1-amino-1,3,3,5,5-pentamethylcyclohexane(neramexane), 1-amino-1,3,5,5-tetramethyl-3-ethylcyclohexane, 1-amino-1,5,5-trimethyl-3,3-diethylcyclohexane, 1-amino-1,5,5-trimethyl-cis-3-ethylcyclohexane, 1-amino-(1S,5 S)cis-3-ethyl-1,5,5-trimethylcyclohexane, 1-amino-1,5,5-trimethyl-trans-3-ethylcyclohexane, 1-amino-(1R,5S)trans-3-ethyl-1,5,5-trimethylcyclohexane, 1-amino-1-ethyl-3,3,5,5-tetramethylcyclohexane, 1-amino-1-propyl-3,3,5,5-tetramethylcyclohexane, N-methyl-1-amino-1,3,3,5,5-pentamethylcyclohexane, N-ethyl-1-amino-1,3,3,5,5-pentamethyl-cyclohexane, N-(1,3,3,5,5-pentamethylcyclohexyl)pyrrolidine, 3,3,5,5-tetramethylcyclohexylmethylamine, 1-amino-1-propyl-3,3,5,5-tetramethylcyclohexane, 1 amino-1,3,3,5(trans)-tetramethylcyclohexane (axial amino group), 3-propyl-1,3,5,5-tetramethylcyclohexylamine semihydrate, 1-amino-1,3,5,5-tetramethyl-3-ethylcyclohexane, 1-amino-1,3,5-trimethylcyclohexane, 1-amino-1,3-dimethyl-3-propylcyclohexane, 1-amino-1,3(trans),5(trans)-trimethyl-3(cis)-propylcyclohexane, 1-amino-1,3-dimethyl-3-ethylcyclohexane, 1-amino-1,3,3-trimethylcyclohexane, cis-3-ethyl-1 (trans)-3(trans)-5-trimethylcyclohexamine, 1-amino-1,3(trans)-dimethylcyclohexane, 1,3,3-trimethyl-5,5-dipropylcyclohexylamine, 1-amino-1-methyl-3(trans)-propylcyclohexane, 1-methyl-3(cis)-propylcyclohexylamine, 1-amino-1-methyl-3(trans)-ethylcyclohexane, 1-amino-1,3,3-trimethyl-5(cis)-ethylcyclohexane, 1-amino-1,3,3-trimethyl-5(trans)-ethylcyclohexane, cis-3-propyl-1,5,5-trimethylcyclohexylamine, trans-3-propyl-1,5,5-trimethylcyclohexylamine, N-ethyl-1,3,3,5,5-pentamethylcyclohexylamine, N-methyl-1-amino-1,3,3,5,5-pentamethylcyclohexane, 1-amino-1-methylcyclohexane, N,N-dimethyl-1-amino-1,3,3,5,5-pentamethylcyclohexane, 2-(3,3,5,5-tetramethylcyclohexyl)ethylamine, 2-methyl-1-(3,3,5,5-tetramethylcyclohexyl)propyl-2-amine, 2-(1,3,3,5,5 -pentamethylcyclohexyl-1)-ethylamine semihydrate, N-(1,3,3,5,5-pentamethylcyclohexyl)-pyrrolidine, 1-amino-1,3(trans),5(trans)trimethylcyclohexane, 1-amino-1,3(cis),5(cis)-trimethylcyclohexane, 1-amino(1R,SS)trans-5-ethyl-1,3,3-trimethylcyclohexane, 1-amino-(1S,SS)cis-5-ethyl-1,3,3-trimethylcyclohexane, 1-amino-1,5,5-trimethyl-3(cis)-isopropyl-cyclohexane, 1-amino-1,5,5-trimethyl-3(trans)-isopropyl-cyclohexane, 1-amino-1-methyl-3(cis)-ethyl-cyclohexane, 1-amino-1-methyl-3(cis)-methyl-cyclohexane, 1-amino-5,5-diethyl-1,3,3-trimethyl-cyclohexane, 1-amino-1,3,3,5,5-pentamethylcyclohexane, 1-amino-1,5,5-trimethyl-3,3-diethylcyclohexane, 1-amino-1-ethyl-3,3,5,5-tetramethylcyclohexane, N-ethyl-1-amino-1,3,3,5,5-pentamethylcyclohexane, N-(1,3,5-trimethylcyclohexyl)pyrrolidine or piperidine, N-[1,3(trans),5(trans)-trimethylcyclohexyl]pyrrolidine or piperidine, N-[1,3(cis),5(cis)-trimethylcyclohexyl]pyrrolidine or piperidine, N-(1,3,3,5-tetramethylcyclohexyl)pyrrolidine or piperidine, N-(1,3,3,5,5-pentamethylcyclohexyl)pyrrolidine or piperidine, N-(1,3,5,5-tetramethyl-3-ethylcyclohexyl)pyrrolidine or piperidine, N-(1,5,5-trimethyl-3,3-diethylcyclohexyl)pyrrolidine or piperidine, N-(1,3,3-trimethyl-cis-5-ethylcyclohexyl)pyrrolidine or piperidine, N-[(1S,SS)cis-5-ethyl-1,3,3-trimethylcyclohexyl]pyrrolidine or piperidine, N-(1,3,3-trimethyl-trans-5-ethylcyclohexyl)pyrrolidine or piperidine, N-[(1R,SS)trans-5-ethyl, 3,3-trimethylcyclohexyl]pyrrolidine or piperidine, N-(1-ethyl-3,3,5,5-tetramethylyclohexyl)pyrrolidine or piperidine, N-(1-propyl-3,3,5,5-tetramethylcyclohexyl)pyrrolidine or piperidine, N-(1,3,3,5,5-pentamethylcyclohexyl)pyrrolidine, their optical isomers, diastereomers, enantiomers, hydrates, their pharmaceutically acceptable salts, and mixtures thereof. One memantine analog is neramexane (1-amino-1,3,3,5,5-pentamethylcyclohexane), which is described, e.g., in U.S. Pat. No. 6,034,134.

Certain memantine analogs of general formula (I) include the case where three axial alkyl substituent, e.g., Rₚ, Rᵣ and R₅ all together form a bridgehead to yield compounds (so called 1-aminoadamantanes) illustrated by the formulae IIb-IId below:

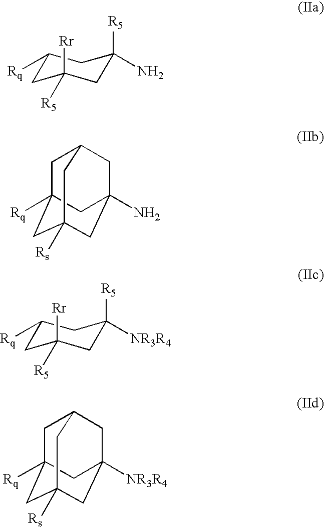

Certain memantine analogs of formula (1) wherein n+m=0, U, V, W, X, Y and Z form a cyclohexane ring, and one or both of R₃ and R₄ are independently joined to the cyclohexane ring via alkylene bridges formed through Rₚ, R_{q}, Rᵣ, Rₛ or R₅ are represented by the following formulas IIIa-IIIc: 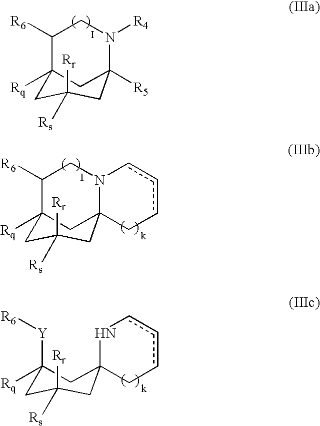
where R_{q}, Rᵣ, Rₛ, Rᵣ and R₅ are as defined above for formula (I), R₆ is hydrogen, linear or branched C₁-C₆ alkyl, linear or branched C₂-C₆ alkenyl, linear or branched C₂-C₆ alkynyl, aryl, substituted aryl or arylalkyl Y is saturated or may combine with R₆ to form a carbon-hydrogen bond with the ring carbon to which it is attached, l=0 or 1 and k=0, 1 or 2 and ------ represents a single or double bond.

Examples of 1-aminocyclohexane derivatives that can be employed in the methods, compositions, and kits of the invention include 1-amino adamantane and its derivatives selected from the group consisting of 1-amino-3-phenyl adamantane, 1-amino-methyl adamantane, 1-amino-3-ethyl adamantane, 1-amino-3-isopropyl adamantane, 1-amino-3-n-butyl adamantane, 1-amino-3,5-diethyl adamantane, 1-amino-3,5-diisopropyl adamantane, 1-amino-3,5-di-n-butyl adamantane, 1-amino-3-methyl-5-ethyl adamantane, 1-N-methylamino-3,5-dimethyl adamantane, 1-N-ethylamino-3,5-dimethyl adamantane, 1-N-isopropylamino-3,5-dimethyl adamantane, 1-N,N-dimethyl-amino-3,5-dimethyl adamantane, 1-N-methyl-N-isopropyl-amino-3-methyl-5-ethyl adamantane, 1-amino-3-butyl-5-phenyl adamantane, 1-amino-3-pentyl adamantane, 1-amino-3,5-dipentyl adamantane, 1-amino-3-pentyl-5-hexyl adamantane, 1-amino-3-pentyl-5-cyclohexyl adamantane, 1-amino-3-pentyl-5-phenyl adamantane, 1-amino-3-hexyl adamantane, 1-amino-3,5-dihexyl adamantane, 1-amino-3-hexyl-5-cyclohexyl adamantane, 1-amino-3-hexyl-5-phenyl adamantane, 1-amino-3-cyclohexyl adamantane, 1-amino-3,5-dicyclohexyl adamantane, 1-amino-3-cyclohexyl-5-phenyl adamantane, 1-amino-3,5-diphenyl adamantane, 1-amino-3,5,7-trimethyl adamantane, 1-amino-3,5-dimethyl-7-ethyl adamantane, 1-amino-3,5-diethyl-7-methyl adamantane, 1-N-pyrrolidino and 1-N-piperidine derivatives, 1-amino-3-methyl-5-propyl adamantane, 1-amino-3-methyl-5-butyl adamantane, 1-amino-3-methyl-5-pentyl adamantane, 1-amino-3-methyl-5-hexyl adamantane, 1-amino-3-methyl-5-cyclohexyl adamantane, 1-amino-3-methyl-5-phenyl adamantane, 1-amino-3-ethyl-5-propyl adamantane, 1-amino-3-ethyl-5-butyl adamantane, 1-amino-3-ethyl-5-pentyl adamantane, 1-amino-3-ethyl-5-hexyl adamantane, 1-amino-3-ethyl-5-cyclohexyl adamantane, 1-amino-3-ethyl-5-phenyl adamantane, 1-amino-3-propyl-5-butyl adamantane, 1-amino-3-propyl-5-pentyl adamantane, 1-amino-3-propyl-5-hexyl adamantane, 1-amino-3-propyl-5-cyclohexyl adamantane, 1-amino-3-propyl-5-phenyl adamantane, 1-amino-3-butyl-5-pentyl adamantane, 1-amino-3-butyl-5-hexyl adamantane, 1-amino-3-butyl-5-cyclohexyl adamantane, their optical isomers, diastereomers, enantiomers, hydrates, N-methyl, N,N-dimethyl, N-ethyl, N-propyl derivatives, their pharmaceutically acceptable salts, and mixtures thereof.

The compounds of formulas IIb and IId, including memantine, may be prepared by alkylation of halogenated adamantanes, preferably bromo- or chloroadamantanes. The di- or tri-substituted adamantanes may be obtained by additional halogenation and alkylation procedures. The amino group is introduced either by oxidation with chromiumtrioxide and bromination with HBr or bromination with bromine and reaction with formamide followed by hydrolysis. The amino function can be alkylated according to generally-accepted methods. Methylation can, for example, be effected by reaction with chloromethyl formate and subsequent reduction. The ethyl group can be introduced by reduction of the respective acetamide. For more details on synthesis see, e.g., U.S. Pat. Nos. 5,061,703 and 6,034,134.

Other memantine analogs are described by formula IV: 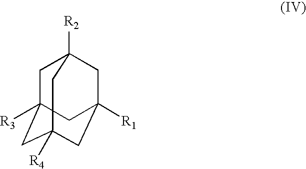
wherein R₁ is NHC(O)R₅, C(O)NHR₅, (CR₅R₆)ₙNR₅R₆ or (CR₅R₆)ₙCO₂R₅; n is an integer ranging from 0 to 4; R₂, R₃ and R₄ are each independently selected from the group consisting of H, fluoro, C₁-C₆ alkyl, and hydroxy; and each R₅ and R₆ is independently H or C₁-C₆ alkyl.

Memantine analogs of formula IV include methyl-3-fluoro-5-hydroxyadamantane-1-carboxylate; fluoroadamantane-1-carboxylic acid; 3,5-difluoro-adamantan-1-ylamine; 3,5-difluoroadamantane-1-carboxylic acid; 3-fluoroadamantan-1-ylamine; methyl-3,5-difluoro-7-hydroxyadamantane-1-carboxylate; 3,5,7-trifluoroadamantane-1-carboxylic acid; 3,5,7-trifluoroadamantan-1-ylamine; and the pharmaceutically acceptable salts of the foregoing compounds.

Still other memantine analogs are described by formula V: 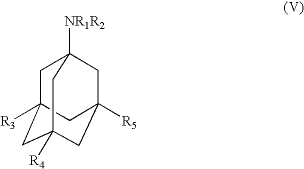
wherein each of R₁ and R₂ is independently hydrogen or a straight or branched C₁-C₆ alkyl or, in conjunction with N, a heterocyclic radical with 5 or 6 ring C atoms; each of R₃ and R₄ is independently hydrogen, a straight or branched C₁-C₆ alkyl, a C₅ or C₆ cycloalkyl, or phenyl; and R₅ is hydrogen or a straight or branched C₁-C₆ alkyl, or a pharmaceutically-acceptable acid addition salt thereof.

Memantine analogs of formula IV include 1-amino adamantane, 1-amino-3-phenyl adamantane, 1-amino-methyl-adamantane, 1-amino-3-ethyl adamantane, 1-amino-3-isopropyl adamantane, 1-amino-3-n-butyl adamantane, 1-amino-3,5-diethyl adamantane, 1-amino-3,5-diisopropyl adamantane, 1-amino-3,5-di-n-butyl adamantane, 1-amino-3-methyl-5-ethyl adamantane, 1-N-methylamino-3,5-dimethyl adamantane, 1-N-ethylamino-3,5-dimethyl adamantane, 1-N-isopropylamino-3,5-dimethyl adamantane, 1-N,N-dimethyl-amino-3,5-dimethyl adamantane, 1-N-methyl-N-isopropyl-amino-3-methyl-5-ethyl adamantane, 1-amino-3-butyl-5-phenyl adamantane, 1-amino-3-pentyl adamantane, 1-amino-3,5-dipentyl adamantane, 1-amino-3-pentyl-5-hexyl adamantane, 1-amino-3-pentyl-5-cyclohexyl adamantane, 1-amino-3-pentyl-5-phenyl adamantane, 1-amino-3-hexyl adamantane, 1-amino-3,5-dihexyl adamantane, 1-amino-3-hexyl-5-cyclohexyl adamantane, 1-amino-3-hexyl-5-phenyl adamantane, 1-amino-3-cyclohexyl adamantane, 1-amino-3,5-dicyclohexyl adamantane, 1-amino-3-cyclohexyl-5-phenyl adamantane, 1-amino-3,5-diphenyl adamantane, 1-amino-3,5,7-trimethyl adamantane, 1-amino-3,5-dimethyl-7-ethyl adamantane, 1-amino-3,5-diethyl-7-methyl adamantane, 1-N-pyrrolidino and 1-N-piperidine derivatives, 1-amino-3-methyl-5-propyl adamantane, 1-amino-3-methyl-5-butyl adamantane, 1-amino-3-methyl-5-pentyl adamantane, 1-amino-3-methyl-5-hexyl adamantane, 1-amino-3-methyl-5-cyclohexyl adamantane, 1-amino-3-methyl-5-phenyl adamantane, 1-amino-3-ethyl-5-propyl adamantane, 1-amino-3-ethyl-5-butyl adamantane, 1-amino-3-ethyl-5-pentyl adamantane, 1-amino-3-ethyl-5-hexyl adamantane, 1-amino-3-ethyl-5-cyclohexyl adamantane, 1-amino-3-ethyl-5-phenyl adamantane, 1-amino-3-propyl-5-butyl adamantane, 1-amino-3-propyl-5-pentyl adamantane, 1-amino-3-propyl-5-hexyl adamantane, 1-amino-3-propyl-5-cyclohexyl adamantane, 1-amino-3-propyl-5-phenyl adamantane, 1-amino-3-butyl-5-pentyl adamantane, 1-amino-3-butyl-5-hexyl adamantane, 1-amino-3-butyl-5-cyclohexyl adamantane, their N-methyl, N,N-dimethyl, N-ethyl, N-propyl derivatives and their acid addition compounds.

Still other memantine analogs are described by formula VIa or formula VIb. 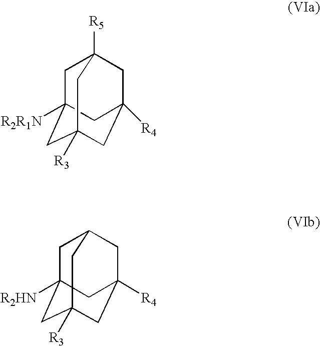
wherein R₁ is H, alkyl, heteroalkyl, aryl, heteroaryl, C(O)OR₆ or C(O)R₆; R₂ is H, alkyl, heteroalkyl, aryl, heteroaryl, C(O)OR₆, or C(O)R₆; R₃ is H, alkyl, heteroalkyl, aryl or heteroaryl; R₄ is H, alkyl, heteroalkyl, aryl or heteroaryl; R₅ is OR₇, alkyl-OR₇, or heteroalkyl-OR₇; R₆ is alkyl, heteroalkyl, aryl, or heteroaryl. R₇ is NO₂, C(O)R₆, C(O)alkyl-ONO₂, or C(O)heteroalkyl-ONO₂. The following substituents are preferred: R₁ and R₂ are H; R₃ and R₄ are H or alkyl; and R₇ is NO₂ or C(O)alkyl-ONO₂. Methods of making these compounds are described, for example, in U.S. Pat. No. 6,620,845.

Memantine analogs of formula VIa or VIb include 1-acetamido-3,5-dimethyl-7-hydroxyadamantane, 1-amino-3,5-dimethyl-7-hydroxyadamantane hydrochloride, 1-tert-butylcarbamate-3,5-dimethyl-7-hydroxy-adamantane, 1-tert-butylcarbamate-3,5-dimethyl-7-nitrate-adamantane, 1-amino-3,5-dimethyl-7-nitrateadamantane hydrochloride, 1-acetamido-3,5-dimethyl-7-nitrateadamantane, 1,1-dibenzylamino-3,5-dimethyl-7-hydroxy-adamantane, 1-amino-3,5-dimethyl-7-acetoxyadamantane hydrochloride, 1-(benzyloxycarbonyl)amino-3,5-dimethyl-7-hydroxyadamantane, 1-(benzyloxycarbonyl)amino-3,5-dimethyl-7-(3-bromopropylcarbonyloxy)adamantane, 1-(benzyloxycarbonyl)amino-3,5-dimethyl-7-(3-nitratepropylcarbonyloxy)adamantane, 1-Acetamido-3,5-dimethyl-7-carboxylic acidadamantane, 1-acetamido-3,5-dimethyl-7-hydroxymethyladamantane, 1-amino-3,5-dimethyl-7-hydroxymethyladamantane hydrochloride, 1-(benzyloxycarbonyl)amino-3,5-dimethyl-7-hydroxymethyl adamantane, 1-(benzyloxycarbonyl)amino-3,5-dimethyl-7-nitratemethyl-adamantane, 1-amino-3,5-dimethyl-7-nitratemethyladamantane hydrobromide, and 1-acetamido-3,5-dimethyl-7-nitratemethyl-adamantane.

Memantine analogs also include N-(1-adamantyl) diethylamine, N-(3-methyl-1-adamantyl)isopropylamine, N-(3,5-dimethyl-1-adamantyl) ethylmethylamine, N-(1-adamantyl)morpholine, N-(3,5,7-trimethly-1-adamantyl) piperidine, N,N′-bis(1-adamantyl)-1,3-propanediamine, N,N′-bis(3-methyl-1-adamantyl)-1,10-decanediamine, N,N′-bis(3,5,7-trimethyl-1-adamantyl)-1,6-hexanediamine, N-(1-adamantyl)cyclohexylamine, N-(1-adamantyl) cyclooctylamine, N-(1-adamantyl)-α-furfurylamine, N-(3-methyl-1-adamantyl)-β-thienylamine, N-(3,5,7-trimethyl-1-adamantyl)-α-furfurylamine, N-(1-adamantyl)-β-thienylamine, N-β-(2-pyridyl)ethyl-1-adamantylamine, N-(3,5-dimethyl-1-adamantyl)-5-phenylpentylamine, bis-adamantylamine, bis(3-methyl-1-adamantyl)amine, bis(3,5-dimethyl-1-adamantyl)amine, N-(1-adamantyl) dodecylamine, N-(1-adamantyl)-N′-phenylpiperazine, N-(1-adamantyl) piperazine, N-(1-adamantyl) aniline, N-(1-adamantyl)benzylamine, N-(1-adamantyl)phenethylamine, N-(1-adamantyl) homoveratylamine, bis(3,5,7-trimethyl-1-adamantyl)amine, N-(3,5,7-trimethyl-1-adamantyl)-1-adamantylamine, 1-aminoadamantane, and N-(3,5,7-trimethyl-1-adamantyl)-N′-phenylpiperazine.

Memantine analogs also include adatanserin, tromantadine, amantanium bromide, rimantadine, somantadine, adapalene, N-1-adamantyl-N′-cyclohexyl-4-morpholinecarboxamidine, dopamantine, adaprolol maleate, (−)—N-(2-(8-methyl-1,4-benzodioxan-2-ylmethylamino)ethyl)adamantane-1-carboxamide, N-(1-adamantyl)-N′,N′-(1,5-(3-(4(5)-1H-imidazolyl-pentanediyl))) formamidine, adamantoyl-Lys-Pro-Tyr-Ile-Leu, 1-(2-pyridyl)-4-(1-methyl-2-(1-adamantylamino)ethyl)piperazine, adafenoxate, (1R,3S)-3-(1-adamantyl)-1-aminomethyl-3,4-dihydro-5,6-dihydroxy-1H-2-benzopyran, adamantylamide L-Ala-L-isoGlu, 2-adamantylamino-benzoic acid, N(alpha)-(1-adamantanesulphonyl)-N-(4-carboxybenzoyl)-L-lysyl-alanyl-L-valinal, 4-acylamino-1-aza-adamantane, L-leucyl-D-methionyl-glucyl-N-(2-adamantyl)-L-phenylalanylamide, Tyr-(D)-Met-Gly-Phe-adamantane, 1-N-(p-bromobenzoyl)methyladamantylamine, 4-butyl-1,2-dihydro-5-((1-adamantanecarbonyl)oxy)-1,2-diphenyl-3H-pyrazol-3-one, N(alpha)-(1-adamantanesulphonyl)-N(epsilon)-succinyl-L-lysyl-L-prolyl-L-valinal, and the amantadine salt of N-acetyl-DL-phenylalanine.

Memantine analogs also include (2-Hydroxy-adamantan-2-yl)-acetic acid ethyl ester, (2-Methyl-adamantan-2-yloxy)-acetic acid, (2-Piperidin-1-yl-adamantan-2-yl)-methylamine, (4-Adamantan-1-yl)-thiazol-2-ylamine, (4-Adamantan-1-yl-phenoxy)-acetic acid (4-Tricyclo[3.3.1.13,7]decan-1-yl-phenoxy-acetic acid), (Adamantan-1-ylmethoxy)-acetic acid, (Adamantan-1-yloxy)-acetic acid, (Adamantan-1-ylsulfanyl)-acetic acid, (Tricyclo[3.3.1.13,7]decan-1-carbonyl-3-aminophenyl-amide), [3-(3,4-Dimethylphenyl)-adamantan-1-yl]-methylamine, 1-(1-Adamantyl)ethyl(2-nitro-5-piperazinophenyl)amine, 1-(1-Adamantyl)ethyl(5-chloro-2-nitrophenyl)amine, 1-(1-Adamantyl)ethylamine Hydrochloride, 1-(4-Hexahydro-1-pyrazinyl-3-nitrophenylcarboxamido)-3,5-dimethyladamantane, 1-(4-Hexahydro-1-pyrazinyl-3-nitrophenylcarboxamido)-adamantane, 1,3-Adamantanediacetic Acid, 1,3-Adamantanedicarboxamide, 1,3-Adamantanedicarboxylic Acid, 1,3-Adamantanedimethanol, 1,3-Dibromoadamantane, 1,3-Dihydroxyadamantane (1,3-Adamantanediol), 1,3-Dimethyladamantane, 1,4-Dibromoadamantane, 1-[1-(4-Hexahydro-1-pyrazinyl-3-nitrophenylcarboxamido)-ethyl]adamantane, 1-Acetamidoadamantane, 1-Adamantan-1-yl-2-methyl-propan-1-one, 1-Adamantan-1-yl-2-phenyl-ethanone, 1-Adamantan-1-yl-3-methyl-butan-1-one, 1-Adamantan-1-yl-3-phenyl-propan-1-one, 1-Adamantan-1-yl-butan-1-one, 1-Adamantan-1-yl-butan-2-one, 1-Adamantan-1-yl-propan-1-one, 1-Adamantan-1-yl-propan-2-one, 1-Adamantanamine, 1-Adamantanamine Hydrochloride, 1-Adamantanamine Sulfate, 1-Adamantaneacetic Acid, 1-Adamantaneacetyl Chloride, 1-Adamantanecarbonitrile, 1-Adamantanecarbonyl Chloride, 1-Adamantanecarboxamide, 1-Adamantanecarboxylic Acid, 1-Adamantaneethanol, 1-Adamantanemethanol, 1-Adamantanemethylamine, 1-Adamantanol (1-Hydroxyadamantane), 1-Adamantyl Bromomethyl Ketone, 1-Adamantyl Methyl Ketone, 1-Amino-3-hydroxy-adamantane hydrochloride, 1-Aminoadamantane sulfate (Bis[1-Aminotricyclo(3.3.1.1.3.7)decane]sulfate), 1-Bromo-3,5-dimethyladamantane, 1-Bromoadamantane, 1-Chloro-3,5-dimethyladamantane, 1-Chloroadamantane, 1-Hydroxy-3,5-dimethyladamantane, 1-Hydroxy-3-amino-5,7-dimethyladamantane hydrochloride, 1-Hydroxy-3-nitro-5,7-dimethyladamantane, 1-Isocyanato-adamantane (1-Isocyanato-tricyclo[3.3.1.13,7]decane), 1-Nitro-3,5-dimethyladamantane, 2-(1-Adamantyl)-4,5-dichloropyridazin-3(2H)-one (4,5-Dichloro-2-tricyclo[3.3.1.13,7]decan-1-yl-2H-pyridazin-3-one), 2-(1-Adamantyl)-5-(chloromethyl)-1,3-thiazole (5-Chloromethyl-2-tricyclo[3.3.1.13,7]decan-1-yl-thiazole), 2-(4-Hexahydro-1-pyrazinyl-3-nitrophenylcarboxamido)-adamantane, 2-(Adamantan-1-ylamino)-ethanol (2-(Tricyclo[3.3.1.13,7]decan-1-yl amino)-ethanol), 2-(Adamantan-1-ylthio)-ethanamine(2-(Tricyclo[3.3.1.13,7]decan-1-ylsulfanyl)-ethylamine), 2-(Adamantan-2-ylamino)-ethanol, 2-[(Adamantan-1-ylmethyl)-amino]-ethanol hydrochloride, 2-Adamantan-1-yl-ethylamine, 2-Adamantanamine Hydrochloride, 2-Adamantanol, 2-Adamantanone (2-Hydroxyadamantane), 2-Adamantanone Oxime, 2-Aminoadamantane Hydrochloride (2-Adamantanamine HCl), 2-Bromoadamantane, 2-Ethyl-2-adamantanol, 2-Methyl-2-Adamantanol, 2-Methyl-2-adamantyl acrylate, 2-Piperidin-1-yl-adamantane-2-carbonitrile, 3-(3,4-Dimethyl-phenyl)-adamantane-1-carboxylic acid, 3-(Adamantan-1-yl)-3-oxo-propionitrile, 3-(Adamantan-1-yl)-4-hydroxy-5-methoxy-benzoic acid, 3-(Adamantan-1-ylsulfanyl)-[1,2,4]-thiadiazol-5-ylamine(3-(Tricyclo[3.3.1.13,7]decan-1-ylsulfanyl)-1,2,4-thiadiazol-5-ylamine), 3-(Adamantan-1-ylsulfanyl)-propylamine, 3,5-Dimethyl-1-adamantanol, 3-Adamantan-1-yl-3-oxo-propionic acid ethyl ester (Tricyclo[3.3.1.13,7]decane-1-propanoic acid, β-oxo-ethyl ester), 3-Adamantan-1-yl-4-methoxy-benzoic acid (4-Methoxy-3-tricyclo[3.3.1.13,7]decan-1-yl-benzoic acid), 3-Hydroxyadamantane-1-carboxylic Acid, 3-Noradamantanecarboxylic Acid, 4,4′-(1,3-Adamantanediyl)diphenol, 4-Adamantan-1-yl-1,2,3-thiadiazole (4-Tricyclo[3.3.1.13,7]dec-1-yl-1,2,3-thiadiazole), 4-Adamantan-1-yl-2-aminophenol (2-Amino-4-tricyclo[3.3.1.13,7]decan-1-yl-phenol), 4-Adamantan-1-yl-5-ethyl-thiazol-2-ylamine, 4-Adamantan-1-yl-5-isopropyl-thiazol-2-ylamine, 4-Adamantan-1-yl-5-methyl-thiazol-2-ylamine, 4-Adamantan-1-yl-5-phenyl-thiazol-2-ylamine, 4-Aza-tricyclo[4.3.1.13,8]undecan-5-one, 4-Aza-tricyclo[4.3.1.13,8]undecane, 5′-Methylspiro[adamantan-2,2′-[1,3]-dioxane]5′-carboxylic acid, 5′-Methylspiro[adamantan-2,2′-[1,3]-dioxane]-5′-amine, 5-Adamantan-1-yl-[1,3,4]-oxadiazole-2-thiol (2-Thiol-5-tricyclo[3.3.1.13,7]dec-1-yl-1,3,4-oxadizol), 5-Adamantan-1-yl-2H-pyrazole-3-carboxylic acid methyl ester, 5-Adamantan-1-yl-2-methoxy-benzoic acid (2-Methoxy-5-tricyclo[3.3.1.13,7]decan-1-yl-benzoic acid), 5-Adamantan-1-yl-2-methyl-furan-3-carboxylic acid (5-Tricyclo[3.3.1.13,7]decan-1-yl-furan-3-carboxylic acid), 5-Adamantan-1-yl-2-methyl-furan-3-carboxylic acid methyl ester (5-Tricyclo[3.3.1.13,7]decan-1-yl-furan-3-carboxylic acid methyl ester), 5-Adamantan-1-yl-2-methyl-phenylamine(2-Methyl-5-tricyclo[3.3.1.13,7]decan-1-yl-phenylamine), 5-Adamantan-1-yl-3-ethyl-isoxazole-4-carboxylic acid, 5-Adamantan-1-yl-3-methyl-isoxazole-4-carboxylic acid, 5-Adamantan-1-yl-furan-2-carboxylic acid (5-Tricyclo[3.3.1.13,7]decan-1-yl-furan-2-carboxylic acid), 5-Adamantan-1-yl-furan-2-carboxylic acid methyl ester (5-Tricyclo[3.3.1.13,7]decan-1-yl-furan-2-carboxylic acid methyl ester), 5-Chloro-2-nitrophenyl(adamantan-2-yl)amine, 5-Hydroxy-2-adamantanone, Adamantan-1-yl-methylamine, Adamantan-2-ylidene-acetonitrile, Adamantane, Adamantane-1-carbonyl isothiocyanate (Tricyclo[3.3.1.13,7]decane-1-carbonyl isothiocyanate), Adamantane-1-carbothioic acid amide(Tricyclo[3.3.1.13,7]decane-1-carbothioic acid amide), Adamantane-1-carboxylic acid (3-amino-phenyl)-amide, Adamantane-1-carboxylic acid (4-amino-2-methoxy-phenyl)-amide (Tricyclo[3.3.1.13,7]decan-1-carbonyl-2-methoxy-3-aminophenyl-amide), Adamantane-1-carboxylic acid (4-amino-phenyl)-amide (Tricyclo[3.3.1.13,7]decan-1-carbonyl-4-aminophenyl-amide), Adamantane-1-sulfinyl chloride, Congressane, Dimethyl 1,3-Adamantanedicarboxylate, Dimethyl-1,3-Adamantanedicarboxylate, Ethyl 1-Adamantanecarboxylate, Methyl 1-Adamantanecarboxylate, N-(1-Adamantyl)ethylenediamine, N-(1-Adamantyl)urea, N-(2-Adamantyl)-N-(4-bromophenyl)amine, N-(Adamantan-2-yl)-N-(2-chloro-ethyl)-amine hydrochloride, N2-(5-hexahydro-1-pyrazinyl-2-nitrophenyl)adamantan-2-yl-amine, N-Adamantan-1-oyl-piperazine, N-Adamantan-1-yl-2-amino-benzamide(2-Amino-N-tricyclo[3.3.1.13,7]decan-1-yl-benzamide), N-Formyl-1-amino-3,5-dimethyladamantane, N-Methyl-(Adamantan-1-yl)methylamine, and p-(1-Adamantyl)phenol.

Additional Agents

If desired, the patient may receive additional therapeutic regimens in combination with memantine or a memantine analog. For example, therapeutic agents may be administered with the agent or agents described herein at concentrations known to be effective or under investigational study for such therapeutic agents. Agents useful to treat a neurodegenerative disease include the following: compounds that correct aberrant SMN protein splicing or protein levels; calcium antagonists such as nimodipine; sodium channel blockers such as fosphenyloin, sipatrigine, and lubeluzole; caspase inhibitors such as p35, ZVAD, and crmA; neuroimmunophilins; amino acids such as taurine and adenosine and other adenosine-based neuroprotectants; competitive and noncompetitive glutamate antagonists such as phencyclidine, ketamine, dizocilpine, dextromethorphan, magnesium, selfotel, MDL 104,653 (3-phenyl-4-hydroxy-7-chloroquinolin-2(1H)-one) and gavestinel; other agents that protect against glutamate-induced toxicity such as TRO 17416 (Trophos SA), TRO 19622 (Trophos SA), and the glutamate receptor agonist TCH-346 (dibenzo[b,f]oxepin-10-ylmethyl-prop-2-ynylamine); benzothiazole class members such as riluzole; free radical scavengers and agents that reduce nitric oxide-related toxicity such as NXY-059 (disodium 2,4-disulfophenyl-N-tert-butylnitrone), lipoic acid, quercitin, peroxynitrite, and lubeluzole; inhibitors of apoptosis such NAIP; growth and trophic factors such as nerve growth factor and glial cell line-derived neurotrophic factor; agents that lower intracellular calcium levels; GABAα receptor activators such as clomethiazole; inhibitors of Rho kinase such as BA-1016 (BioAxone Therapeutic Inc.); Rho antagonists such as Cethrin (BioAxone Therapeutic Inc.; U.S. Pat. No. 6,855,688); protein-based therapeutics such as RI-820; agents that stabilize the neuronal membrane potential; neurosteroids such as allopregnanolone and dehydroepiandrosterone; anti-inflammatory or analgesic agents such as nonsteroidal anti-inflammatory agents; tetracycline compounds such as minocycline; neuropeptides such as neuropeptides (opioid peptides, thyreoliberine, neuropeptide Y, galanin, VIP/PACAP, hormones such as estrogen and progestin, and caffeine); Co-enzyme Q10; creatinine; hydroxyurea; sodium or phenyl butyrate or other butyrate compounds; HDAC inhibitors such as valproate or valproic acid; aclarubicin; gabapentin; albuterol; quinazolines; aminogylcosides; and salbutamol.

Other agents useful to treat a neurodegenerative disease are epigallocatechin-3-gallate; (R)-(−)-BPAP; 106362-32-7; remacemide; selegiline; 4-C1-kynurenine; A-134974; A-366833; A-35380; A-72055; ABS-205; AC-184897; AC-90222; ACEA-1021 (licostinel); ADCI; AEG-3482; AGY-110; AGY-207; AK-275 (vasolex); alaptid; ALE-0540; AM-36; annovis; ampakines; amyloid-inhibiting peptides; AN-1792; andrographolide; APBPI-124; apoptosin; aptiganel; AR-139525; AR-15896 (lanicemine); AR-A-008055; donepezil; AR-R-17779; AR-R18565; ARRY-142886; ARX-2000; ARX-2001; ARX-2002; AS-600292; AS-004509; AS-601245; autovac; axokine; AZ-36041; BA-1016; Bay Q 3111 (BAY-X-9227; N-(2-ethoxyphenyl)-N′-(1,2,3-trimethylpropyl)-2-nitroethene-1,1-diamine); BD-1054; BGC-20-1178; BIMU-8 ((endo-N-8-methyl-8-azabicyclo-(3.2.1)oct-3-yl)-2,3-dihydro-3-isopropyl-2-oxo-1H-benzimidazol-1-carboxamide); BLS-602; BLS-605; BMS-181100 (alpha-(4-fluorophenyl)-4-(5-fluoro-2-pyrimidinyl)-1-piperazine butanol); brasofensine; breflate; BTG-A derivatives; C60 fullerenes; CAS-493 (aloracetam); celecoxib; CEP-1347; CEP-3122; CEP-4143; CEP-4186; CEP-751; CERE-20; CGP-35348 (P-(3-aminopropyl)-P-diethoxymethylphosphinic acid); CHF-2060; CNIC-568; CNS-1044; CNS-2103; CNS-5065; coenzyme Q10; CP-132484 (1-(2-aminoethyl)-3-methyl-8,9-dihydropyrano(3,2-e)indole); CP-283097; CPC-304; CX-516; cyclophosphamide; cyclosporin A; dabelotine; DCG-IV (2-(2,3-dicarboxycyclopropyl)glycine); DD-20207; dehydroascorbic acid; dexanabinol; dexefaroxan; dihydroquinolines; diperdipine; dizocilpine; DMP-543; DP-103; DP-109; DP-b99; DPP-225; dykellic acid; E-2101; EAA-404 (midafotel); EAB-318; edaravone; EF-7412; EGIS-7444; EHT-202; eliprodil; emopamil; EP-475; EQA-00 (anapsos); ES-242-1; estrogen or estrogen/progesterone; ethanoanthracene derivatives; F-10981; F-2-CCG-I; FCE-29484A; FCE-29642A; FGF-9; FGF-16; ersofermin; formobactin; FPL-16283; GAG mimetics; galantamine derivatives; galdansetron; ganstigmine; gavestinel; GDNF (liatermine); GGF-2; GKE-841 (retigabine); glialines (throphix); GM-1 ganglioside; GP-14683; GPI-1337; GPI-1485; GR-73632; GR-89696 (methyl 4-((3,4-dichlorophenyl)acetyl)-3-(1-pyrrolidinylmethyl)-1-piperazinecarboxylate fumarate); GSK-3 inhibitors; GT-2342; GT-715; GV-2400; GYKI-52466 (4-(8-methyl-9H-1,3-dioxolo(4,5-h)(2,3)benzodiazepin-5-yl)-benzenamine); HBNF; HF-0220; HP-184 (N-(n-propyl)-3-fluoro-4-pyridinyl-1H-3-methylindol-1-amine hydrochloride); LAPs; IDN-6556; IGF modulators (e.g., neurocrine); igmesine; imidazole derivatives; imidazolyl nitrones; inosine; interferon alpha; interleukin-2-like growth factor; iometopane; ipenoxazone; itameline; KF-17329; KP-102 (alanyl-(2-naphthyl)alanyl-alanyl-tryptophyl-phenylalanyl-lysinamide); KRX-411; KW-6002 (istradefylline; 8-(2-(3,4-dimethoxyphenyl)ethenyl)-1,3-diethyl-3,7-dihydro-7-methyl-1H-purine-2,6-dione); L-687306 (3-(3-cyclopropyl-1,2,4-oxadiazol-5-yl)-1-azabicyclo(2.2.1)heptane); L-687414; L-689560 (trans-2-carboxy-5,7-dichloro-4-(((phenylamino)carbonyl)amino)-1,2,3,4-tetrahydroquinoline); L-701252; lamotrigine; LAU-0501; lazabemide; leteprinim; LIGA-20; LY-178002 (5-((3,5-bis(1,1-dimethylethyl)-4-hydroxyphenyl)methylene)-4-thiazolidinone); LY-233536 (decahydro-6-(2H-tetrazol-5-ylmethyl)-3-isoquinolinecarboxylic acid); LY-235959 (decahydro-6-(phosphonomethyl)-3-isoquinolinecarboxylic acid); LY-274614; LY-302427; LY-354006; LY-354740 (2-aminobicyclo(3.1.0)hexane-2,6-dicarboxylic acid); LY-451395; MCC-257; MCI-225 (4-(2-fluorophenyl)-6-methyl-2-(1-piperazinyl)thieno(2,3-d)pyrimidine); MDL-100748 (4-((carboxymethyl)amino)-5,7-dichloroquinoline-2-carboxylic acid); MDL-101002; MDL-102288; MDL-105519; MDL-27266 (5-(4-chlorophenyl)-4-ethyl-2,4-dihydro-2-methyl-3H-1,2,4-triazol-3-one); MDL-28170 (carbobenzoxyvalylphenylalanine aldehyde); MDL-29951 (3-(4,6-dichloro-2-carboxyindol-3-yl)propionic acid); mecasermin; MEM-1003; mepindolol; metallotexa-phyrins; methylphenylethynylpyridine (MPEP); microalgal compound; milacemide; mirapex (pramipexole); MLN-519; MS-153; MT-5; N-3393; naltrindole derivatives; NAPVSIPQ; NBI-30702; NC-531; neotrofin; neramexane; nerve growth factor gene therapy; neublastin; neurocalc; neurostrol; NLA-715 (clomethiazole); NNC-07-0775; NNC-07-9202 (2,3-dioxo-6-nitro-7-sulfamoylbenzo(f)quinoxaline); noggin; norleu; NOX-700; NPS-1407; NPS-846; NRT-115; NS-1209; NS-1608 (N-(3-(trifluoromethyl)phenyl)-N′-(2-hydroxy-5-chlorophenyl)urea); NS-2330; NS-257; NS-377; NS-638 (2-amino-1-(4-chlorobenzyl)-5-trifluoromethylbenzimidazole); NS-649; NXD-5150; NXY-059; odapipam; olanzapine; ONO-2506; OPC-14117 (7-hydroxy-1-(4-(3-methoxyphenyl)-1-piperazinyl)acetylamino-2,2,4,6-tetramethylindan); P-58; P-9939; PACAP; palmidrol; PAN-811; pan-neurotrophin-1; PBT-1 (clioquinol); PD-132026; PD-150606 (3-(4-iodophenyl)-2-mercapto-(Z)-2-propenoic acid); PD-159265; PD-90780; PDC-008.004; PE21; phenserine; philanthotoxins; piperidine derivatives; PK-11195 (1-(2-chlorophenyl)-N-methyl-N-(1-methylpropyl)-3-isoquinolinecarboxamide); PN-277; PNU-101033E; PNU-157678; PNU-87663; POL-255; posatirelin; PPI-368; PRE-103; propentofylline; protirelin; PRS-211220; PYM-50028; QG-2283; rasagiline; REN-1654; REN-1820; RI-820; riluzole; RJR-1401; Ro-09-2210; rolipram; RPR-104632 (2H-1,2,4-benzothiadiazine-1-dioxide-3-carboxylate acid); RS-100642; S-14820; S-176251; S-34730-1; S-34730; S-18986; S-312-d (methyl 4,7-dihydro-3-isobutyl-6-methyl-4-(nitrophenyl)thieno(2,3-b)-pyridine-5-carboxylate); S-33113-1; sabeluzole; safinamide; SB-271046; SB-277011 (trans-N-(4-(2-(6-cyano-1,2,3,4-tetrahydroisoquinolin-2-yl)ethyl)cyclohexyl)-4-quinolinecarboxamide); SEMAX; SIB-1553A; SIB-1765F (5-ethynyl-3-(1-methyl-2-pyrrolidinyl)pyridine maleate); siclofen; SJA-6017 (N-(4-fluorophenylsulfonyl)-L-valyl-L-leucinal); SKF-74652; SL-34.0026; SLV-308; SNX-482; SP-(V5.2)C; SPC-9766; SPH-1371; SPM-914; SPM-935; SSR-180575; SSR-482073; sumanirole; SUN-C5174; survivins; SYM-2207; T-588 (1-(benzo(b)thiophen-5-yl)-2-(2-(N,N-diethylamino)ethoxy)ethanol hydrochloride); tacrine analogs (ABS-301, ABS-302, ABS-304); talampanel; taltirelin; TAN-950A (2-amino-3-(2,5-dihydro-5-oxo-4-isoxazolyl)propanoic acid); TC-2559; TCH-346; TGP-580; thurinex; TK-14; TP-20; traxoprodil; U-74500A (21-(4-(3,6-bis(diethylamino)-2-pyridinyl)-1-piperazinyl)-16-methylpregna-1,4,9(11)triene-3,20-dione HCl); U-78517F (2-((4-(2,6-di-1-pyrrolidinyl-4-pyrimidinyl)-1-piperazinyl)methyl)-3,4-dihydro-2,5,7,8-tetramethyl-2H-1-benzopyran-6-ol.di-HCl); UK-351666; UK-356464; UK-356297; vanoxerine; VX-799; WAY-855; WIB-63480-2; WIN-67500; WIN-68100; WIN-69211; xaliprodene; YM-90K (6-(1H-imidazol-1-yl)-7-nitro-2,3(1H,4H)-quinoxalinedione); ziconotide; and zonampanel.

In particular embodiments, memantine or an analog thereof is administered in combination with a second agent selected from alosetron, amrinone, ascorbic acid, indoprofen, ubenimex, and guanidinium-containing compounds such as guanfacine, guanethidine, creatine, guamecycline, guanabenz, guanadrel, guanoxabenz, and guanoxan. Guanfacine (N-aminoiminomethyl)-2,6-dichlorobenzeneacetamide) is an alpha adrenergic receptor agonist. It's chemical structure and methods of making it are described in U.S. Pat. No. 3,632,645. Guanethidine ([2-(hexahydro-1(2H)-azocinyl)ethyl]guanidine) is an antihypertensive norepinephrine-depleting agent. It's chemical structure and methods of making it are described in U.S. Pat. No. 2,928,829. Analogs of any of the foregoing can also be used in the compositions, methods, and kits of the invention. Such analogs are described in U.S. Pat. Nos. 2,928,829; 3,247,221; 3,547,951; 3,591,636; 3,632,645; GB 1019120; and GB 1042207, each of which is hereby incorporated by reference. Ubenimex ([(2S,3R)-3-amino-2-hydroxy-4-phenylbutanoyl]-L-leucine) and analogs thereof are described in U.S. Pat. Nos. 4,029,547, 4,052,449, 4,189,604, and include (2S,3R)-3-amino-2-hydroxy-4-phenylbutanoyl-(R)-leucine; (2S,3S)-3-amino-2-hydroxy-4-phenylbutanoyl-(R)-leucine; (2S,3S)-3-amino-2-hydroxy-4-phenylbutanoyl-(S)-leucine; (2S,3R)-3-amino-2-hydroxy-4-p-nitrophenylbutanoyl-(S)-leucine; (2S,3R)-3-amino-2-hydroxy-4-phenylbutanoyl-(S)-valine; (2S,3R)-3-amino-2-hydroxy-4-phenylbutanoyl-(S)-norvaline; (2S,3R)-3-amino-2-hydroxy-4-phenylbutanoyl-(S)-methionine; (2S,3R)-3-amino-2-hydroxy-4-phenylbutanoyl-(S)-isoleucine; (2S,3R)-3-amino-2-hydroxy-4-phenylbutanoyl-(S)-norleucine; (2RS,3RS)-3-amino-2-hydroxy-4-p-chlorophenylbutanoyl-(S)-leucine; (2RS,3RS)-3-amino-2-hydroxy-4-o-chlorophenylbutanoyl-(S)-leucine; (2RS,3RS)-3-amino-2-hydroxy-4-p-methylphenylbutanoyl-(S)-leucine; (2S,3R)-3-amino-2-hydroxy-4-p-aminophenylbutanoyl-(S)-leucine; (2RS,3RS)-3-amino-2-hydroxy-4-p-hydroxyphenylbutanoyl-(S)-leucine; (2S,3R)-3-amino-2-hydroxy-4-p-hydroxyphenylbutanoyl-(S)-leucine; (2S,3R)-3-amino-2-hydroxy-4-phenylbutanoic acid; (2S,3R)-3-amino-2-hydroxy-4-p-hydroxyphenylbutanoic acid; (2RS,3RS)-3-amino-2-hydroxy-4-phenylbutanoic acid; (2RS,3RS)-3-amino-2-hydroxy-4-p-hydroxyphenylbutanoic acid; (2RS,3RS)-3-amino-2-hydroxy-4-p-chlorophenylbutanoic acid; (2RS,3RS)-3-amino-2-hydroxy-4-o-chlorophenylbutanoic acid; (2RS,3RS)-3-amino-2-hydroxy-4-p-methylphenylbutanoic acid; and (2RS,3RS)-3-amino-2-hydroxy-4-p-benzyloxyphenylbutanoic acid. Alosetron analogs included granisetron, azasetron, tropisetron, ramosetron, ondansetron, lerisetron, zacopride, cilansetron, itasetron, indisetron, dolasetron, Ro-93777, YM-114, talipexole, fabesetron, tropisetron, mirtazapine, ramosetron, N-3389, E-3620, lintopride, KAE-393, and mosapride. Certain alosetron are described in U.S. Pat. Nos. 5,360,800 and 5,344,927, and European Patent Publication Nos. EP 0189002, EP 0361317, and EP 0306323. Amrinone and analogs thereof are described in U.S. Pat. Nos. 4,004,012 and 4,072,746.

If more than one agent is employed, therapeutic agents may be delivered separately or may be admixed into a single formulation. When agents are present in different pharmaceutical compositions, different routes of administration may be employed. Routes of administration for the various embodiments include, but are not limited to, topical, transdermal, and systemic administration (such as, intravenous, intramuscular, intrathecal, epidural, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration. Desirably, the agent of the invention and additional therapeutic agents are administered within at least 1, 2, 4, 6, 10, 12, 18, 24 hours, 3 days, 7 days, or 14 days apart. The dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds. Optionally, any of the agents of the combination may be administered in a low dosage or in a high dosage, each of which is defined herein.

The therapeutic agents of the invention may be admixed with additional active or inert ingredients, e.g., in conventional pharmaceutically acceptable carriers. A pharmaceutical carrier can be any compatible, non-toxic substance suitable for the administration of the compositions of the present invention to a mammal. Pharmaceutically acceptable carriers include, for example, water, saline, buffers and other compounds described for example in the Merck Index, Merck & Co., Rahway, N.J. Slow release formulation or a slow release apparatus may be also be used for continuous administration.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Other formulations particularly suited for administration of drugs to infants or children are also contemplated. In one embodiment, memantine, amantadine, or an analog thereof is formulated as a flavored suspension. Such flavored suspensions are well known in the art and are described, for example, in U.S. Pat. No. 6,793,935 and U.S. Patent Application Publication Nos. 2005-0233001 and 2005-0013835. Suitable flavors include cherry, orange, and the like. In another embodiment, memantine, amantadine, or an analog thereof is formulated as a dissolving powder that is optionally flavored and suitable for dissolving in water, milk, formula, and/or fruit juice (e.g., apple juice, orabge juice, or grape juice). In still another embodiment, memantine, amantadine, or an analog thereof is formulated as a powder suitable for sprinkling on a variety of foods (e.g., baby food mixes, yogurt, cereal, etc.) without interacting with the food. In one example, a composition of the invention is in the form of polymer-coated taste-masked beads.

Dosages

Generally, when administered to a human, the dosage of any of the agents of the combination of the invention will depend on the nature of the agent, and can readily be determined by one skilled in the art. Typically, such dosage is normally about 0.001 mg to 2000 mg per day, desirably about 1 mg to 1000 mg per day, and more desirably about 5 mg to 500 mg per day. Dosages up to 200 mg per day may be necessary. Administration of each agent in the combination can, independently, be one to four times daily for one day to one year, and may even be for the life of the patient. Chronic, long-term administration will be indicated in many cases.

Additional Applications

If desired, the compounds of the invention may be employed in mechanistic assays to determine whether other combinations, or single agents, are as effective as the combination in treating neurodegenerative diseases (e.g., SMA) using assays generally known in the art, examples of which are described herein. For example, candidate compounds may be tested, alone or in combination (e.g., with an agent that is useful for treating a neurodegenerative disease, such as those described herein) and applied to fibroblasts derived from patients diagnosed as having SMA. After a suitable time, these cells are examined for SMN protein levels. An increase in SMN protein levels identifies a candidate compound or combination of agents as an effective agent to treat a neurodegenerative disease.

The agents of the invention are also useful tools in elucidating mechanistic information about the biological pathways involved in SMN protein regulation. Such information can lead to the development of new combinations or single agents for treating SMA or another neurodegenerative disease. Methods known in the art to determine biological pathways can be used to determine the pathway, or network of pathways affected by contacting cells (e.g., fibroblasts or motorneurons) with the compounds of the invention. Such methods can include, analyzing cellular constituents that are expressed or repressed after contact with the compounds of the invention as compared to untreated, positive or negative control compounds, and/or new single agents and combinations, or analyzing some other activity of the cell such as enzyme activity. Cellular components analyzed can include gene transcripts, and protein expression. Suitable methods can include standard biochemistry techniques, radiolabeling the compounds of the invention (e.g., ¹⁴C or ³H labeling), and observing the compounds binding to proteins, e.g. using 2D gels, gene expression profiling. Once identified, such compounds can be used in in vivo models (e.g., a mouse model for SMA) to further validate the tool or develop new agents or strategies to treat neurodegenerative diseases.

Application to Other Diseases

Memantine may act by increasing transcription, modifying splicing, inducing translational read-through, and/or increasing protein stability, and thus may, alone or in combination, be useful for treating other diseases that are caused by low expression of a gene. Such diseases include cancers that can be sent into growth arrest by the up-regulation of tumor suppressor genes such as p53 and transcriptional targets of the retinoblastoma protein. Other diseases that may be treating by administration of memantine include diseases caused by low gene expression due to premature stop codons, such as Duchenne muscular dystrophy and cystic fibrosis. Diseases that arise from splicing defects include familial isolated growth hormone deficiency, type II (IGHD II), Frasier syndrome and other disorders that result from abnormal expression of the Wilms tumor suppressor gene (WT1), frontotemporal dementia and Parkinsonism linked to chromosome 17 (FTDP-17), Hutchinson-Gilford progeria syndrome (HGPS), myotonic dystrophy, retinitis pigmentosa, atypical cystic fibrosis, neurofibromatosis type I (NF1), Fanconi's anemia, and breast cancer susceptibility at the BRCA1/BRCA2 loci. Diseases that may benefit to therapies that increase protein stability include hematological malignancies and solid tumors, stroke, and ischemia.

Small Molecule Stimulators of SMN Protein

There are a variety of mechanisms that could lead to increases in SMN protein concentration; such mechanisms include transcription initiation and elongation, pre-mRNA splicing, mRNA decay and stability, translation initiation and elongation, and protein degradation. All of these mechanisms can be surveyed simultaneously by screening for small molecules that increase the amount of SMN protein in SMA patient fibroblasts. Subsequent to identifying compounds with this property, it will be possible to identify which of these specific mechanisms is responsible for each compound's effect.

We set out to identify single agents that increase the concentration of the survival motor neuron (SMN) protein in mammalian cells. The copy number of the human SMN2 gene, and by extension the amount of SMN protein, has been found to inversely correlate with SMA disease severity in both humans and mice. Thus, compounds that increase the amount of SMN protein in cells are likely to be effective therapies for patients with SMA.

### EXAMPLE 1

One method for monitoring SMN protein levels in cells is through use of a cytoblot assay, in which cells are fixed and probed with an antibody against a target protein of interest. We have used a cytoblot assay to determine the concentration of SMN protein in SMA patient fibroblasts, and have memantine and amantadine as compounds that increase SMN protein levels.

Using the cytoblot assay, we can clearly distinguish SMN protein levels in patient (GM03813) versus carrier fibroblast cells (GM03814). We performed parallel GAPDH cytoblot assays and verified that the difference in signal does not reflect a difference in cell number. Parallel western blots show a similar distinction between the two fibroblast lines, and also demonstrate that the antibody used (from BD Biosciences) is highly specific for SMN protein.

SMN Cytoblot Protocol

The SMN cytoblot protocol is described below.

Day 1

1. Plate 50 μl per well of 300,000 cells/ml (15,000 cells per well) into Nunc white 384-well opaque-bottomed plates.
2. Incubate plates at 37° C., 5% CO₂ overnight.
   Day 2
1. Aspirate media from wells using Tecan plate washer.
2. Fix cells by adding 20 μl/well of cold methanol (kept in −20° C. freezer).
3. Incubate plates in 4° C. refrigerator for 10 minutes.
4. Aspirate methanol using Tecan plate washer.
5. Add 50 μl/well of primary Ab solution, (BD anti-SMN Ab diluted to 125 ng/ml in 10% fetal goat serum PBST), using multi-drop.
6. Seal plates and incubate overnight at room temperature.
   Day 3
1. After 16-20 hrs incubation, wash plates with PBST 2× using Tecan plate washer.
2. Add 50 μl of secondary Ab solution, (Santa Cruz goat anti-Mouse-HRP diluted to 67 ng/ml in 10% fetal goat serum PBST).
3. Seal plates and incubate 2 hrs at room temperature.
4. Wash plates with PBST 2× using Tecan plate washer.
5. Add 50 μl/well 10% fetal goat serum PBST.
6. Incubate in 4° C. refrigerator for 10 minutes.
7. Aspirate plates using plate washer and add ECL luminescence solution, 20 μl/well.
8. Measure luminescence on plate reader.
   Automated Scoring

We scored each compound based on the maximum signal-to-noise ratio (SNR) found among its dose curves. These scores are based on edge-corrected SMN data, using only plates that passed quality control. The untreated level on each plate was found by taking the median of the untreated wells, and for each treated well we calculated a log(T/U) ratio. Each curve was generated in triplicate on each plate, so each point on a compound's dose curve was obtained by determining the median for the replicate points. Each ratio thus has an error estimate from the scatter between the triplicate data points (1.5 times the median absolute deviation from the median). For each dose curve, we calculated the SNR for each data point log(T/U)/error, and chose the maximum point as our indicator of SMN induction activity. Since this is a signal-to-noise score, scores much greater than one indicate significant activity, assuming normal statistics. We decided to use a cutoff signal-to-noise ratio of 6. This selection represents a considerable enrichment towards visually-selected compounds. We identified 100 out of 2000 compounds producing scores>6. This included 13 of the 28 visual selections.

SMN Assay Visual Selection Criteria

1. Type 1: Raw hits
   (a) Increased SMN signal in multiple wells
   (b) Consistency between replicate wells
   (c) Signal looks significant relative to surrounding wells and overall quality of plate
   (d) Signal not created by edge correction
   (e) Additional weight given to hits that replicate in retest
   (f) Plates that did not pass quality control may have been used as additional reinforcement if the signal was very strong relative to the noise
2. Type 2: normalized hits
   (a) SMN signal flat or slightly up, while Alamar blue data indicates toxicity
   (b) Additional weight given to hits that replicate in retest
   (c) Plates that did not pass quality control may have been used as additional reinforcement if the signal was very strong relative to the noise
3. Type 3 reversal of curve hits

### EXAMPLE 2

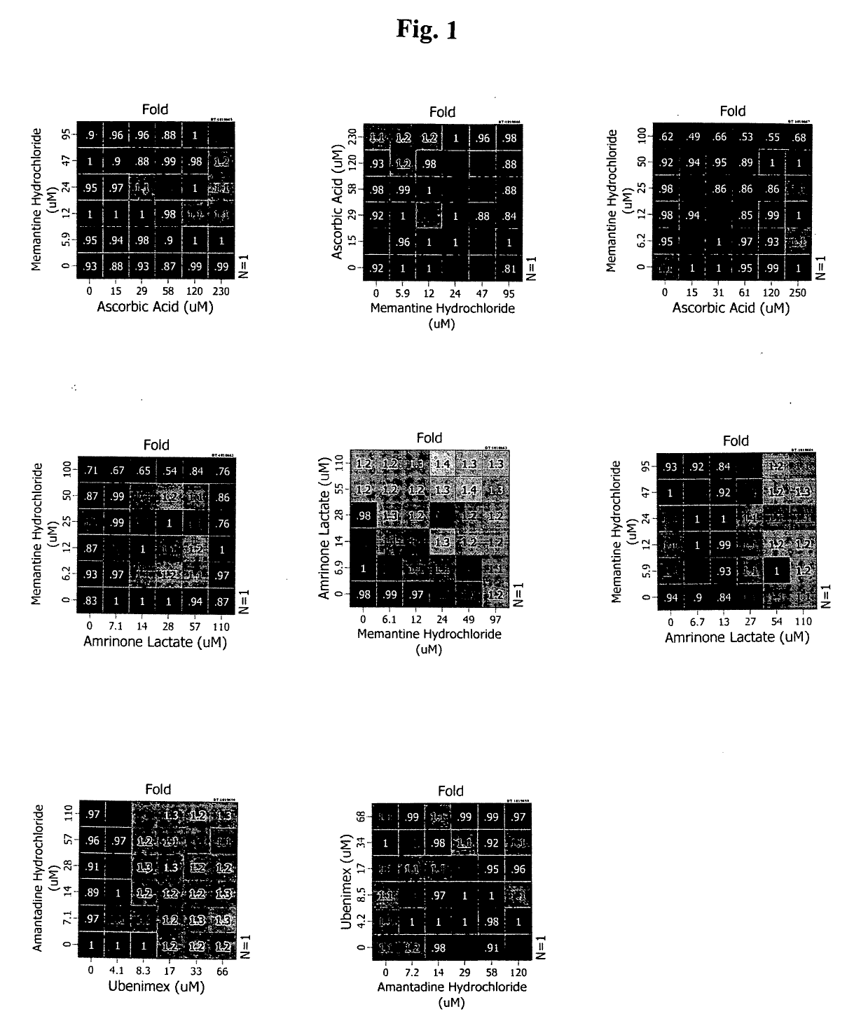

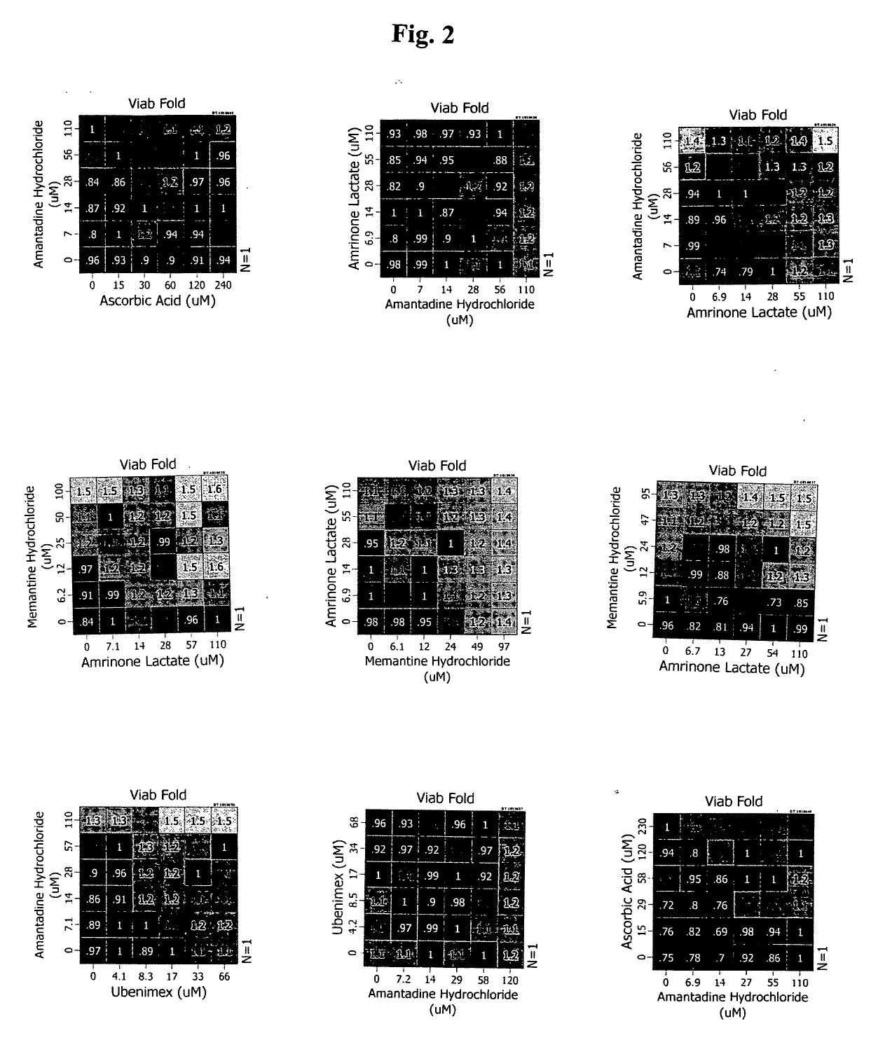

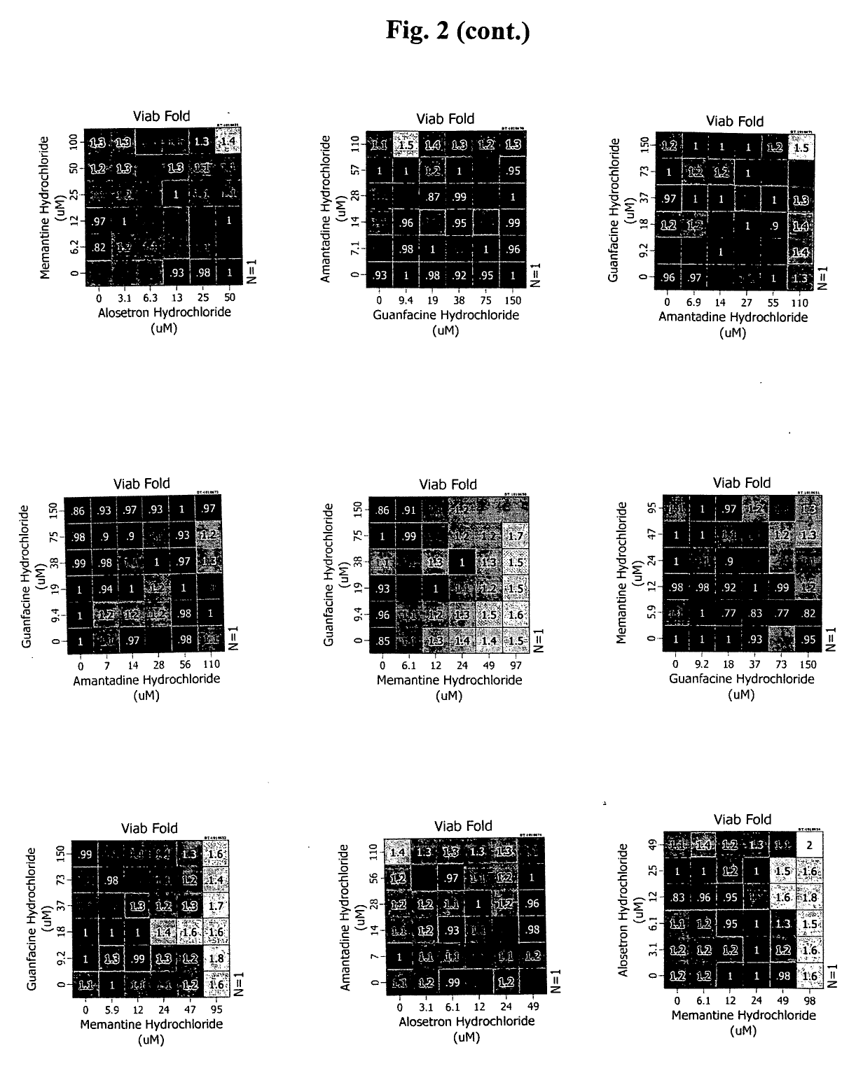

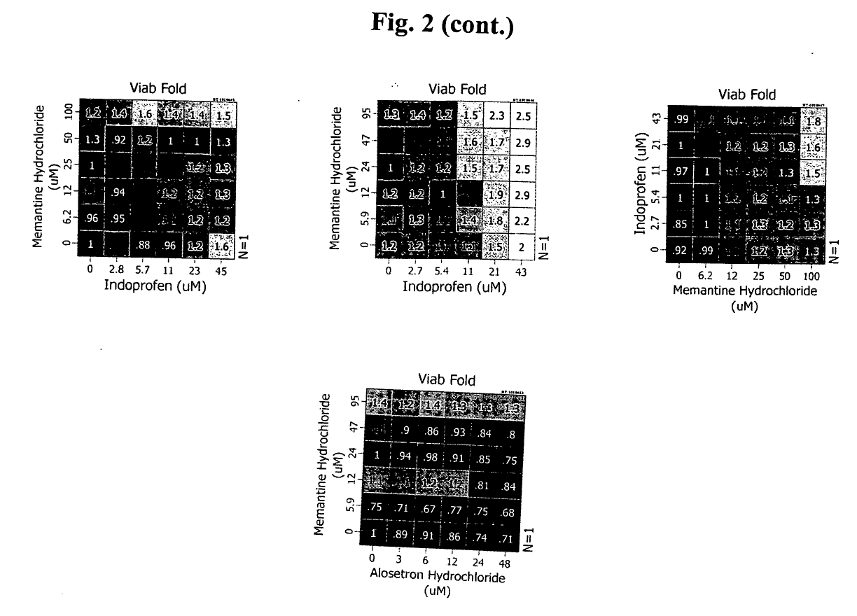

The following combinations were assayed to determine their ability to increase levels of SMN protein in GM03813 fibroblast cells: ascorbic acid and memantine; ascorbic acid and amantadine; ubenimex and amantadine; amrinone and memantine; amrinone and amantadine; guanfacine and memantine; gunafacine and amantadine; alosetron and memantine; alosetron and amantadine; and indoprofen and memantine. The results are shown in FIGS. 1 and 2.

Methods

Day 1

Trypsinize confluent GM03813 fibrobast cells (passage 3-10) from Corning T-175 Tissue Culture flasks. Dilute cells to 89,000 cells/ml in MEM. Using multi-drop, add 45 μl/well to white 384-well opaque bottomed tissue culture treated plates. Incubate plates at 37° C., 5% CO₂ overnight.

Using PlateMate, add 60 μl per well to clear 384-well plates; one plate per master plate to be used.

Using “Two Drug M×M” program, transfer 3 μl from master to dilution plate (20× dilution) and 5 μl from dilution to assay plate (10× dilution) for a total 200× dilution of compounds. Create two daughter assay plates per combination of X and Y master plate. Spin plates briefly (˜30 seconds at 1000 RPM). Return assay plates to incubator for 72 hr incubation.

Day 4

ATP lite 1-step Addition: Reconstitute powder with assay buffer according to product instructions. Using PlateMate, add 50 μl per well to appropriate assay plates. Protect plates from light for ten minutes and place plates on orbital plate shaker (at least 700 RPM) for two minutes. Read plates on Wallac readers using SMAF_Lumi protocol.

Cell Fixation and Primary Antibody Addition: Remove remaining plates from incubator. Wash plates 2× using Tecan Plate washer with PBS, 0.1% Tween 20. Using PlateMate, add cold methanol (stored in −20° C. freezer) to plates, 30 μl well. Incubate plates in 4° C. refrigerator for ten minutes. Repeat 2× washing using Tecans. Using PlateMate, add anti-SMN or antibody to plates, 40 μl/well. Seal plates and incubate at room temperature overnight.

Day 5

Secondary Antibody Addition and Luminescence: Wash plates 2× as above. Using PlateMate, add secondary antibody solution to plates, 30 μl/well. Seal plates and incubate at room temperature for two hours. After incubation wash plates 4× as above. Using PlateMate, add streptavidin-HRP solution to plates, 3011 per well. Incubate 1.5 hours. Wash plates 3× as above. Using PlateMate, add Amersham ECL solution to plates, 20 μl/well. After ECL addition and before plate reading, dark adapt plates for approximately 3 minutes in order to eliminate luminescent signal from the plate itself. Measure luminescence on Wallac.

Data Analysis

Combination data were scored both as absolute SMN fold induction (FIG. 1) and as “viability controlled” SMN fold induction (SMN/ATP) (FIG. 2). SMN fold induction was calculated as SMN(T−B)/SMN(U−B) where “T” is the signal from treated cells, “B” is plate-specific background, and “U” is the signal from untreated cells. Viability controlled fold induction was calculated as (SMN fold induction)/(ATP fold induction), where ATP fold induction is calculated in the same manner as SMN fold induction, or: ATP(T−B)/ATP(U−B).

Combination data matrices were compared to the highest single agent (HSA) and Loewe additivity (ADD) models. HSA volume (HSA vol) and additivity volume (ADD vol) scores were determined for each matrix. The volume score is the sum across the entire matrix of the excess of the observed signal compared to the signal predicted by the model. Thus a score of 1 indicates that the matrix performs at the level predicted by the model.

### EXAMPLE 3

Memantine and amantadine were identified as normalized hits using the methods described above (i.e., as compounds that increase SMN per viable cell). Treated and control wells were assayed for SMN protein by SMN cytoblot, while cell number was estimated by Alamar blue, which evaluates metabolic activity and thus provides an estimate of viable cell number. Hits were chosen for follow-up if compound treatment resulted in SMN levels were increased relative to the estimate of cell number by Alamar blue.

Hits from the primary cytoblot assay were confirmed by repeated cytoblot assays using an ATP viability assay (Perkin Elmer ATP-lite 1-step kit). The normalized effects per cell number were calculated as follows: Normalized fold change=(fold change SMN)/(fold change cell number control), where fold change=(treated−background)/(untreated−background).

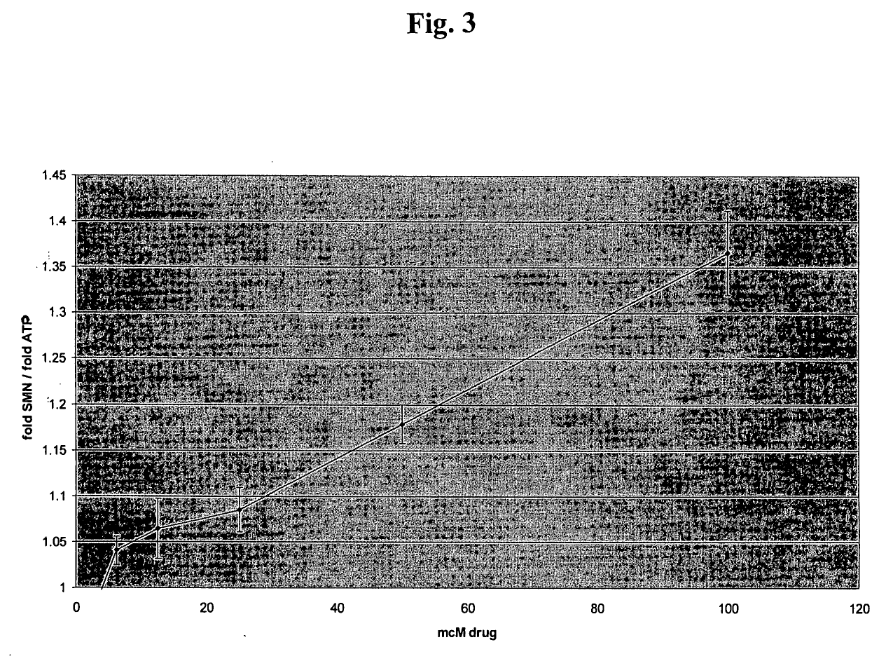

The data shown in FIG. 3 use ATP assay as viability control as described in protocol. Data are mean and standard error from 57-62 data points per dose.

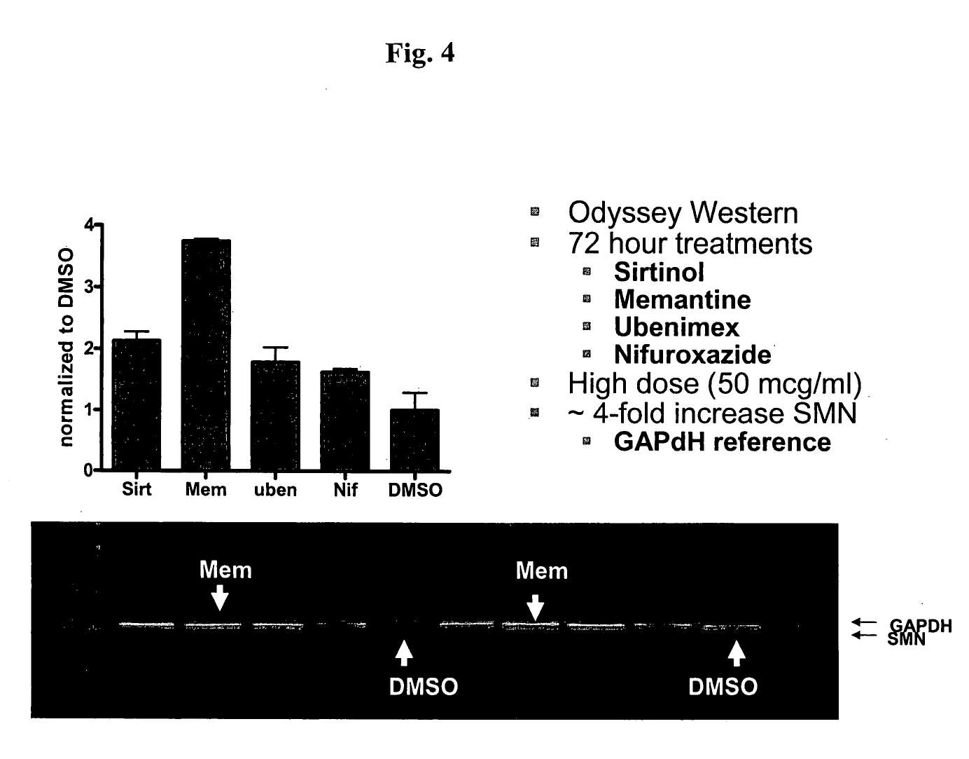

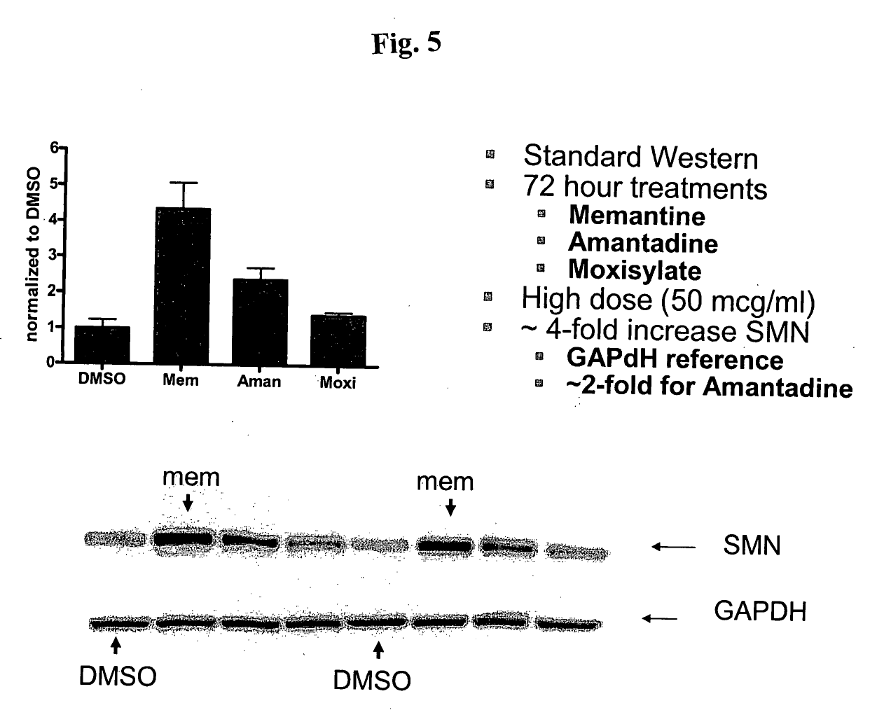

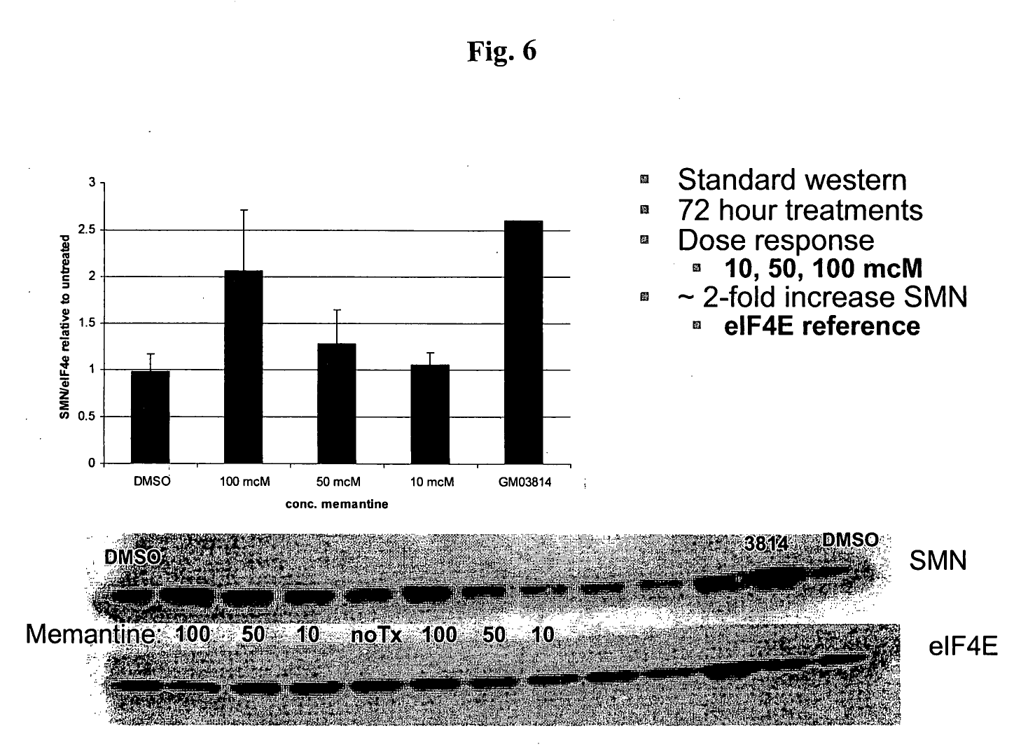

Effects on SMN protein levels were also confirmed by western blots. Cells were treated with compound or vehicle for 72 hours. Cell lysates were prepared and analyzed by western blot. SMN protein levels were quantified by densitometry and normalized to an internal control protein (GAPdH or EIF4e) (FIGS. 4-6). The SMN/control ratios shown in FIGS. 4-6 depict the fold change relative to vehicle (DMSO) control.

### Other Embodiments

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

What is claimed is:

1. A method of treating spinal muscular atrophy (SMA) in a patient in need thereof, said method comprising administering to said patient an effective amount of an agent selected from memantine, amantadine, and analogs thereof.

2. The method of claim 1, wherein said SMA is SMN-related SMA.

3. The method of claim 1, wherein said agent is administered orally

4. The method of claim 1, wherein said agent is administered systemically.

5. The method of claim 1, wherein said agent is memantine.

6. The method of claim 1, wherein said agent is amantadine.

7. The method of claim 1, further comprising administering to said patient a second agent selected from alosetron, amrinone, ascorbic acid, guanfacine, indoprofen, ubenimex, and agents useful for treating a neurodegenerative disease, wherein the first and second agents are administered simultaneously or within 28 days of each other, in amounts that together are effective to treat said SMA.

8. The method of claim 7, wherein said first and second agents are administered within 14 days of each other.

9. The method of claim 8, wherein said first and second agents are administered within 7 days of each other.

10. The method of claim 9, wherein said first and second agents are administered within 24 hours of each other.

11. The method of claim 7, wherein said second agent is administered orally.

12. The method of claim 7, wherein said second agent is administered systemically.

13. The method of claim 1, wherein said memantine is administered in a range from about 5-100 mg/day.

14. The method of claim 13, wherein said memantine is administered in a range from about 10-40 mg/day.

15. The method of claim 14, wherein said memantine is administered at a dose of about 20 mg/day.

16. A composition comprising:
(a) a first agent selected from memantine, amantadine, and analogs thereof, and
(b) a second agent selected from alosetron, amrinone, ascorbic acid, guanfacine, indoprofen, ubenimex, and agents useful for treating a neurodegenerative disease, wherein said first and second agents are present in amounts that together are effective to treat SMA.

17. The composition of claim 16, wherein said composition is formulated for oral administration.

18. The composition of claim 16, wherein said composition is formulated for systemic administration.

19. A kit comprising:
(i) an agent selected from memantine, amantadine, and analogs thereof; and
(ii) instructions for administering said agent to a patient having SMA.

20. A kit comprising:
(i) a composition comprising (a) a first agent selected from memantine, amantadine, and analogs thereof, and (b) a second agent selected from alosetron, amrinone, ascorbic acid, guanfacine, indoprofen, ubenimex, and agents useful for treating a neurodegenerative disease; and
(ii) instructions for administering said composition to a patient having SMA.

21. A kit comprising:
(i) a first agent selected from memantine, amantadine, and analogs thereof;
(ii) a second agent selected from alosetron, amrinone, ascorbic acid, guanfacine, indoprofen, ubenimex, and agents useful for treating a neurodegenerative disease; and
(iii) instructions for administering said first and said second agents to a patient having SMA.

22. A method of identifying a combination that may be useful for the treatment of a neurodegenerative disease, said method comprising the steps of:
(a) contacting cells with (i) an agent selected from memantine, amantadine, and analogs thereof and (ii) a candidate compound; and
(b) determining whether the combination of said agent and said candidate compound increases SMN protein levels relative to cells contacted with said agent but not contacted with the candidate compound, wherein an increase in SMN protein levels identifies the combination as a combination useful for the treatment of a neurodegenerative disease.

23. The method of claim 22, wherein said cells are mammalian cells.

24. The method of claim 23, wherein said cells are human cells.

25. The method of claim 22, wherein said cells are derived from a patient diagnosed as having SMA.

26. The method of claim 25, wherein said SMA is SMN-related SMA.

27. A method for increasing SMN protein levels in a patient having SMA, said method comprising administering to said patient an effective amount of an agent selected from memantine, amantadine, and analogs thereof.

28. A method for increasing SMN protein levels in a patient having SMA, said method comprising administering to said patient a first agent selected from memantine, amantadine, and analogs thereof, and a second agent selected from alosetron, amrinone, ascorbic acid, guanfacine, indoprofen, ubenimex, and agents useful for treating a neurodegenerative disease, wherein the first and second agents are administered simultaneously or within 28 days of each other, in amounts that together are sufficient to increase SMN protein levels in said patient.

29. The method of claim 28, wherein said first and second agents are administered within 14 days of each other.

30. The method of claim 29, wherein said first and second agents are administered within seven days of each other.

31. The method of claim 30, wherein said first and second agents are administered within 24 hours of each other.
